(19) United States
(12) Patent Application Publication
(10) Pub. No.: US 2009/0203718 A1
(43) Pub. Date: Aug. 13, 2009

(54) CANCER TREATMENT METHOD

(75) Inventors: David Rusnak, Research Triangle Park, NC (US); Tona Morgan Gilmer, Research Triangle Park, NC (US)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: SmithKline Beecham (Cork) Ltd., Curraghbinny, Carrigaline (IE)

(21) Appl. No.: 12/296,972

(22) PCT Filed: Apr. 13, 2006
(86) PCT No.: PCT/US2007/066478

§ 371 Date: Oct. 13, 2008

Related U.S. Application Data
(60) Provisional application No. 60/791,841, filed on Apr. 13, 2006.

(51) Int. Cl. A61K 31/517; A61P 35/00

(52) U.S. Cl. 514/266.4

(57) ABSTRACT

The present invention relates to a method of treating cancer in a mammal and to pharmaceutical combinations useful in such treatment. In particular, the method relates to a cancer treatment method that includes administering an erb family inhibitor and an IGF-1R inhibitor to a mammal suffering from a cancer.

### BACKGROUND OF THE INVENTION

The present invention relates to a method of treating cancer in a mammal and to pharmaceutical combinations useful in such treatment. In particular, the method relates to a cancer treatment method that includes administering an erbB-2 and/or an EGFR inhibitor with an IGF-1R inhibitor to a mammal suffering from a cancer.

Effective chemotherapy for cancer treatment is a continuing goal in the oncology field. Generally, cancer results from the deregulation of the normal processes that control cell division, differentiation and apoptotic cell death. There is significant interaction among the ErbB family that regulates the cellular effects mediated by these receptors. Six different ligands that bind to EGFR include EGF, transforming growth factor, amphiregulin, heparin binding EGF, betacellulin and epiregulin (Alroy & Yarden, FEBS Letters, 410:83-86, 1997; Burden & Yarden, Neuron, 18: 847-855, 1997; Klapper et al., Proc Natl Acad Sci, 4994-5000, 1999). Heregulins, another class of ligands, bind directly to HER3 and/or HER4 (Holmes et al., Science, 256:1205, 1992; Klapper et al., 1997, Oncogene, 14:2099-2109; Peles et al., Cell, 69:205, 1992). Binding of specific ligands induces homo- or heterodimerization of the receptors within members of the erbB family (Carraway & Cantley, Cell, 78:5-8, 1994; Lemmon & Schlessinger, Trends Biochem Sci, 19:459-463, 1994). In contrast with the other ErbB receptor members, a soluble ligand has not yet been identified for HER2, which seems to be transactivated following heterodimerization. The heterodimerization of the erbB-2 receptor with the EGFR, HER3, and HER4 is preferred to homodimerization (Klapper et al., 1999; Klapper et al., 1997). Receptor dimerization results in binding of ATP to the receptor's catalytic site, activation of the receptor's tyrosine kinase, and autophosphorylation on C-terminal tyrosine residues. The phosphorylated tyrosine residues then serve as docking sites for proteins such as Grb2, Shc, and phospholipase C, that, in turn, activate downstream signaling pathways, including the Ras/MEK/Erk and the PI3K/Akt pathways, which regulate transcription factors and other proteins involved in biological responses such as proliferation, cell motility, angiogenesis, cell survival, and differentiation (Alroy & Yarden, 1997; Burgering & Coffer, Nature, 376:599-602, 1995; Chan et al., Ann Rev Biochem, 68:965-1014, 1999; Lewis et al., Adv Can Res, 74:49-139, 1998; Liu et al., Genes and Dev, 13:786-791, 1999; Muthuswamy et al., Mol&CellBio, 19, 10:6845-6857, 1999; Riese & Stern, Bioessays, 20:41-48, 1998).

The type 1 receptor for insulin-like growth factor (IGF-1R) is a transmembrane receptor with tyrosine kinase activity which binds initially to IGF1 but also to IGF2 and to insulin with lower affinity. The binding of IGF1 to its receptor leads to oligomerization of the receptor, activation of the tyrosine kinase, intermolecular autophosphorylation and phosphorylation of cell substrates (main substrates: IRS1, Shc, and Src). Routinely, IGF-1R, when activated by its ligand, induces mitogenic activity in normal cells. However, IGF-1R also plays an important role in “abnormal” growth. Several clinical reports underline the important role of the IGF-1 pathway in the development of human cancers. IGF-1R is often found overexpressed in many tumor types (prostate, breast, colon, lung, sarcoma, etc.) and its presence is often associated with a more aggressive phenotype. (See U.S. Pat. No. 6,340,674; Macaulay, British Journal of Cancer 1992, 65:311-320) High concentrations of circulating IGF1 correlate strongly with a risk of prostate cancer, lung cancer and breast cancer. In addition, it has been widely documented that IGF-1R is necessary for establishing and maintaining the transformed phenotype in vitro as in vivo (R Baserga, Exp. Cell. Res., 1999, 253, pages 1-6). The kinase activity of IGF-1R is essential to the transforming activity of several oncogenes: EGFR, PDGFR, SV40 virus large T antigen, activated Ras, Raf, and v-Src. The expression of IGF-1R in normal fibroblasts induces a neoplastic phenotype, which can then lead to the formation of a tumor in vivo. The expression of IGF-1R plays an important role in substrate-independent growth. IGF-1R has also been shown to be a protector in apoptosis induced by chemotherapy and radiation, and apoptosis induced by cytokines. In addition, the inhibition of endogenous IGF-1R by a dominant negative, the formation of a triple helix or the expression of an antisense causes suppression of the transforming activity in vitro and a decrease in tumor growth in animal models.

The present inventors proposed that a combination of an IGF-1R kinase inhibitor and GW572016 or another inhibitor of ErbB signaling would provide an improved cancer treatment method. Consequently, it has now been recognized, that a combination of an erb family and IGR-1R inhibitor appears to be more effective than either therapy by itself. Accordingly, the present inventors have now discovered a new method of treating cancer using a novel pharmaceutical combination, which can selectively treat susceptible cancers. Specifically, the novel combination of a dual EGFR/erbB-2 inhibitor and an IGF-1R inhibitor appears to effectively inhibit growth of such tumors and at times the combination of a dual EGFR/erbB-2 inhibitor and an IGF-1R inhibitor may act synergistically.

### SUMMARY OF THE INVENTION

In a first aspect of the present invention, there is provided a method of treating a susceptible cancer in a mammal, comprising: administering to said mammal therapeutically effective amounts of (i) a compound of formula (I)

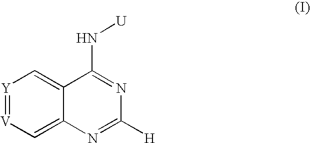

or a salt or solvate thereof;
wherein

### Y is CR¹ and V is N;

### or Y is CR¹ and V is CR²;

R¹ represents a group CH₃SO₂CH₂CH₂NHCH₂—Ar—, wherein Ar is selected from phenyl, furan, thiophene, pyrrole and thiazole, each of which may optionally be substituted by one or two halo, C₁₋₄ alkyl or C₁₋₄ alkoxy groups;
R² is selected from the group comprising hydrogen, halo, hydroxy, C₁₋₄ alkyl, C₁₋₄ alkoxy, C₁₋₄ alkylamino and di[C₁₋₄ alkyl]amino;
U represents a phenyl, pyridyl, 3H-imidazolyl, indolyl, isoindolyl, indolinyl, isoindolinyl, 1H-indazolyl, 2,3-dihydro-1H-indazolyl, 1H-benzimidazolyl, 2,3-dihydro-1H-benzimidazolyl or 1H-benzotriazolyl group, substituted by an R³ group and optionally substituted by at least one independently selected R⁴ group;
R³ is selected from a group comprising benzyl, halo-, dihalo- and trihalobenzyl, benzoyl, pyridylmethyl, pyridylmethoxy, phenoxy, benzyloxy, halo-, dihalo- and trihalobenzyloxy and benzenesulphonyl;
or R³ represents trihalomethylbenzyl or trihalomethylbenzyloxy;
or R³ represents a group of formula

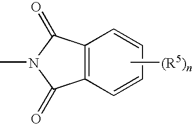

wherein each R⁵ is independently selected from halogen, C₁₋₄ alkyl and C₁₋₄ alkoxy;
and n is 0 to 3;
each R⁴ is independently hydroxy, halogen, C₁₋₄ alkyl, C₂₋₄ alkenyl, C₂₋₄ alkynyl, C₁₋₄ alkoxy, amino, C₁₋₄ alkylamino, di[C₁₋₄ alkyl]amino, C₁₋₄ alkylthio, C₁₋₄ alkylsulphinyl, C₁₋₄ alkylsulphonyl, C₁₋₄ alkylcarbonyl, carboxy, carbamoyl, C₁₋₄ alkoxycarbonyl, C₁₋₄ alkanoylamino, N—(C₁₋₄ alkyl)carbamoyl, N,N-di(C₁₋₄ alkyl)carbamoyl, cyano, nitro and trifluoromethyl; and
(ii) at least one IGF-1R inhibitor.

In a second aspect of the present invention, there is provided a method of treating a susceptible cancer in a mammal, comprising: administering to said mammal therapeutically effective amounts of (i) a compound of formula (II):

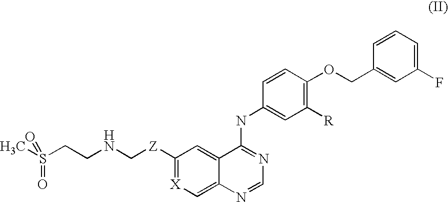

or a salt or solvate thereof, wherein R is —Cl or —Br, X is CH, N, or CF, and Z is thiazole or furan; and
(ii) at least one IGF-1R inhibitor.

In a third aspect of the present invention, there is provided a method of treating a susceptible cancer in a mammal, comprising: administering to said mammal therapeutically effective amounts of (i) a compound of formula (III):

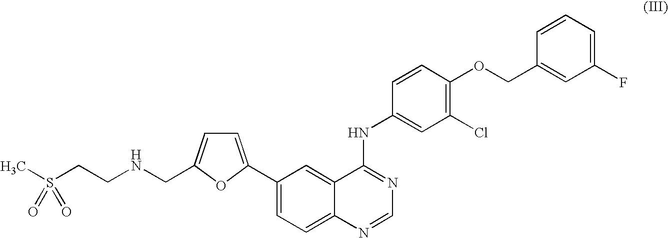

or a salt or solvate thereof; and
(ii) at least one IGF-1R inhibitor.

In a fourth aspect of the present invention, there is provided a cancer treatment combination, comprising: therapeutically effective amounts of (i) a compound of formula (I)

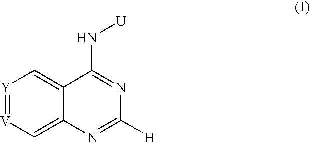

or a salt or solvate thereof;
wherein

### Y is CR¹ and V is N;

### or Y is CR¹ and V is CR²;

R¹ represents a group CH₃SO₂CH₂CH₂NHCH₂—Ar—, wherein Ar is selected from phenyl, furan, thiophene, pyrrole and thiazole, each of which may optionally be substituted by one or two halo, C₁₋₄ alkyl or C₁₋₄ alkoxy groups;
R² is selected from the group comprising hydrogen, halo, hydroxy, C₁₋₄ alkyl, C₁₋₄ alkoxy, C₁₋₄ alkylamino and di[C₁₋₄ alkyl]amino;
U represents a phenyl, pyridyl, 3H-imidazolyl, indolyl, isoindolyl, indolinyl, isoindolinyl, 1H-indazolyl, 2,3-dihydro-1H-indazolyl, 1H-benzimidazolyl, 2,3-dihydro-1H-benzimidazolyl or 1H-benzotriazolyl group, substituted by an R³ group and optionally substituted by at least one independently selected R⁴ group;
R³ is selected from a group comprising benzyl, halo-, dihalo- and trihalobenzyl, benzoyl, pyridylmethyl, pyridylmethoxy, phenoxy, benzyloxy, halo-, dihalo- and trihalobenzyloxy and benzenesulphonyl;
or R³ represents trihalomethylbenzyl or trihalomethylbenzyloxy;
or R³ represents a group of formula

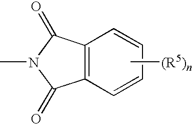

wherein each R⁵ is independently selected from halogen, C₁₋₄ alkyl and C₁₋₄ alkoxy;
and n is 0 to 3;
each R⁴ is independently hydroxy, halogen, C₁₋₄ alkyl, C₂₋₄ alkenyl, C₂₋₄ alkynyl, C₁₋₄ alkoxy, amino, C₁₋₄ alkylamino, di[C₁₋₄ alkyl]amino, C₁₋₄ alkylthio, C₁₋₄ alkylsulphinyl, C₁₋₄ alkylsulphonyl, C₁₋₄ alkylcarbonyl, carboxy, carbamoyl, C₁₋₄ alkoxycarbonyl, C₁₋₄ alkanoylamino, N—(C₁₋₄ alkyl)carbamoyl, N,N-di(C₁₋₄ alkyl)carbamoyl, cyano, nitro and trifluoromethyl; and
(ii) at least one IGF-1R inhibitor.

In a fifth aspect of the present invention, there is provided a cancer treatment combination, comprising: therapeutically effective amounts of (i) a compound of formula (II):

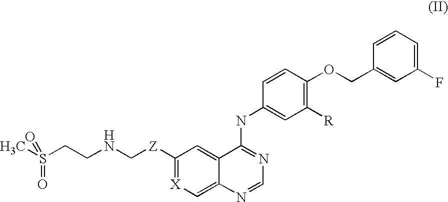

or a salt or solvate thereof, wherein R is —Cl or —Br, X is CH, N, or CF, and Z is thiazole or furan; and
(ii) at least one IGF-1R inhibitor.

In a sixth aspect of the present invention, there is provided a cancer treatment combination, comprising: therapeutically effective amounts of (i) a compound of formula (III):

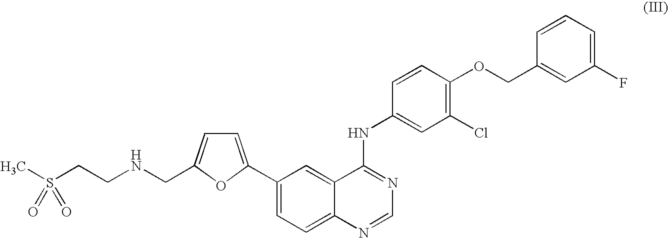

or a salt or solvate thereof; and
(ii) at least one IGF-1R inhibitor.

In a seventh aspect of the present invention, there is provided a cancer treatment combination, comprising: therapeutically effective amounts of (i) a compound of formula (I)

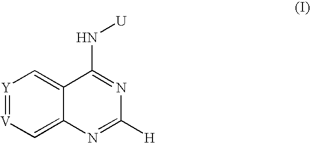

or a salt or solvate thereof;
wherein

### Y is CR¹ and V is N;

### or Y is CR¹ and V is CR²;

R¹ represents a group CH₃SO₂CH₂CH₂NHCH₂—Ar—, wherein Ar is selected from phenyl, furan, thiophene, pyrrole and thiazole, each of which may optionally be substituted by one or two halo, C₁₋₄ alkyl or C₁₋₄ alkoxy groups;
R² is selected from the group comprising hydrogen, halo, hydroxy, C₁₋₄ alkyl, C₁₋₄ alkoxy, C₁₋₄ alkylamino and di[C₁₋₄ alkyl]amino;
U represents a phenyl, pyridyl, 3H-imidazolyl, indolyl, isoindolyl, indolinyl, isoindolinyl, 1H-indazolyl, 2,3-dihydro-1H-indazolyl, 1H-benzimidazolyl, 2,3-dihydro-1H-benzimidazolyl or 1H-benzotriazolyl group, substituted by an R³ group and optionally substituted by at least one independently selected R⁴ group;
R³ is selected from a group comprising benzyl, halo-, dihalo- and trihalobenzyl, benzoyl, pyridylmethyl, pyridylmethoxy, phenoxy, benzyloxy, halo-, dihalo- and trihalobenzyloxy and benzenesulphonyl;
or R³ represents trihalomethylbenzyl or trihalomethylbenzyloxy;
or R³ represents a group of formula

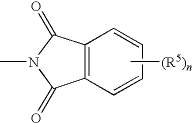

wherein each R⁵ is independently selected from halogen, C₁₋₄ alkyl and C₁₋₄ alkoxy;
and n is 0 to 3;
each R⁴ is independently hydroxy, halogen, C₁₋₄ alkyl, C₂₋₄ alkenyl, C₂₋₄ alkynyl, C₁₋₄ alkoxy, amino, C₁₋₄ alkylamino, di[C₁₋₄ alkyl]amino, C₁₋₄ alkylthio, C₁₋₄ alkylsulphinyl, C₁₋₄ alkylsulphonyl, C₁₋₄ alkylcarbonyl, carboxy, carbamoyl, C₁₋₄ alkoxycarbonyl, C₁₋₄ alkanoylamino, N—(C₁₋₄ alkyl)carbamoyl, N,N-di(C₁₋₄ alkyl)carbamoyl, cyano, nitro and trifluoromethyl; and
(ii) at least one IGF-1R inhibitor;
for use in therapy.

In an eighth aspect of the present invention, there is provided a cancer treatment combination, comprising: therapeutically effective amounts of (i) a compound of formula (II):

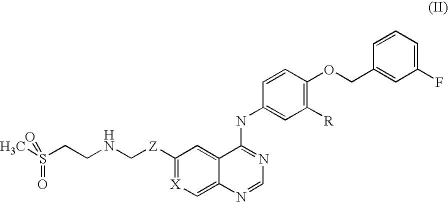

or a salt or solvate thereof, wherein R is —Cl or —Br, X is CH, N, or CF, and Z is thiazole or furan; and
(ii) at least one IGF-1R inhibitor; for use in therapy.

In a ninth aspect of the present invention, there is provided a cancer treatment combination, comprising: therapeutically effective amounts of (i) a compound of formula (III):

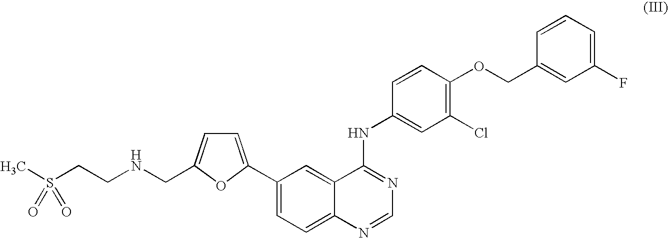

or a salt or solvate thereof; and
(ii) at least one IGF-1R inhibitor; for use in therapy.

In a tenth aspect of the present invention, there is provided a cancer treatment combination, comprising: therapeutically effective amounts of (i) a compound of formula (I)

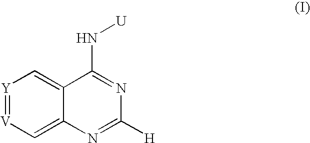

or a salt or solvate thereof;
wherein

### Y is CR¹ and V is N;

### or Y is CR¹ and V is CR²;

R¹ represents a group CH₃SO₂CH₂CH₂NHCH₂—Ar—, wherein Ar is selected from phenyl, furan, thiophene, pyrrole and thiazole, each of which may optionally be substituted by one or two halo, C₁₋₄ alkyl or C₁₋₄ alkoxy groups;
R² is selected from the group comprising hydrogen, halo, hydroxy, C₁₋₄ alkyl, C₁₋₄ alkoxy, C₁₋₄ alkylamino and di[C₁₋₄ alkyl]amino;
U represents a phenyl, pyridyl, 3H-imidazolyl, indolyl, isoindolyl, indolinyl, isoindolinyl, 1H-indazolyl, 2,3-dihydro-1H-indazolyl, 1H-benzimidazolyl, 2,3-dihydro-1H-benzimidazolyl or 1H-benzotriazolyl group, substituted by an R³ group and optionally substituted by at least one independently selected R⁴ group;
R³ is selected from a group comprising benzyl, halo-, dihalo- and trihalobenzyl, benzoyl, pyridylmethyl, pyridylmethoxy, phenoxy, benzyloxy, halo-, dihalo- and trihalobenzyloxy and benzenesulphonyl;
or R³ represents trihalomethylbenzyl or trihalomethylbenzyloxy;
or R³ represents a group of formula

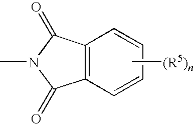

wherein each R⁵ is independently selected from halogen, C₁₋₄ alkyl and C₁₋₄ alkoxy;
and n is 0 to 3;
each R⁴ is independently hydroxy, halogen, C₁₋₄ alkyl, C₂₋₄ alkenyl, C₂₋₄ alkynyl, C₁₋₄ alkoxy, amino, C₁₋₄ alkylamino, di[C₁₋₄ alkyl]amino, C₁₋₄ alkylthio, C₁₋₄ alkylsulphinyl, C₁₋₄ alkylsulphonyl, C₁₋₄ alkylcarbonyl, carboxy, carbamoyl, C₁₋₄ alkoxycarbonyl, C₁₋₄ alkanoylamino, N—(C₁₋₄ alkyl)carbamoyl, N,N-di(C₁₋₄ alkyl)carbamoyl, cyano, nitro and trifluoromethyl; and
(ii) at least one IGF-1R inhibitor;
for use in the preparation of a medicament useful in the treatment of a susceptible cancer.

In an eleventh aspect of the present invention, there is provided a cancer treatment combination, comprising: therapeutically effective amounts of (i) a compound of formula (II):

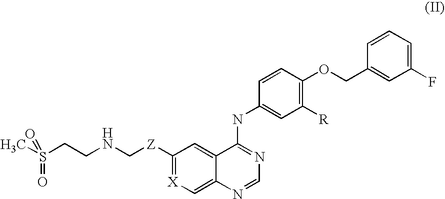

or a salt or solvate thereof, wherein R is —Cl or —Br, X is CH, N, or CF, and Z is thiazole or furan; and
(ii) at least one IGF-1R inhibitor; for use in the preparation of a medicament useful in the treatment of a susceptible cancer.

In a twelvth aspect of the present invention, there is provided a cancer treatment combination, comprising: therapeutically effective amounts of (i) a compound of formula (III):

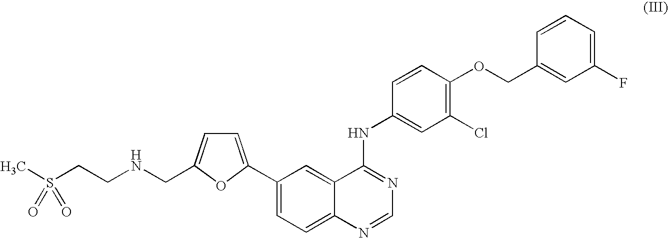

or a salt or solvate thereof; and
(ii) at least one IGF-1R inhibitor; for use in the preparation of a medicament useful in the treatment of a susceptible cancer.

### BRIEF DESCRIPTION OF THE DRAWINGS

FIG. 1 depicts results from BrdU cell proliferation, CelltiterGlo total cell number, Caspase Induction apoptosis, and Roche Cell Death ELISA apoptosis assays after dosing A549 cells with GW572016, GSK621659A, and a 1:1 combination of GW572016 and GSK621659A.

FIG. 2 depicts results from BrdU cell proliferation, CelltiterGlo total cell number, Caspase Induction apoptosis, and Roche Cell Death ELISA apoptosis assays after dosing Colo205 cells with GW572016, GSK621659A, and a 1:1 combination of GW572016 and GSK621659A.

FIG. 3 depicts results from BrdU cell proliferation, CelltiterGlo total cell number, Caspase Induction apoptosis, and Roche Cell Death ELISA apoptosis assays after dosing MDA-MB-468 cells with GW572016, GSK621659A, and a 1:1 combination of GW572016 and GSK621659A.

FIG. 4 depicts a graph showing the response of dosing various cancer cell lines with GW572016, alpha-IF₃ antibody, and a combination of both.

FIG. 5 depicts results from Roche Cell Death ELISA apoptosis assays after dosing various cancer cell lines with GW572016, GSK621659A, and a 1:1 combination of GW572016 and GSK621659A.

### DETAILED DESCRIPTION OF THE INVENTION

As used herein the term “neoplasm” refers to an abnormal growth of cells or tissue and is understood to include benign, i.e., non-cancerous growths, and malignant, i.e., cancerous growths. The term “neoplastic” means of or related to a neoplasm.

As used herein the term “agent” is understood to mean a substance that produces a desired effect in a tissue, system, animal, mammal, human, or other subject. Accordingly, the term “anti-neoplastic agent” is understood to mean a substance producing an anti-neoplastic effect in a tissue, system, animal, mammal, human, or other subject. It is also to be understood that an “agent” may be a single compound or a combination or composition of two or more compounds.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, the term “optionally” means that the subsequently described event(s) may or may not occur, and includes both event(s), which occur, and events that do not occur.

As used herein, the term “solvate” refers to a complex of variable stoichiometry formed by a solute (in this invention, a compounds formulae (I), (II), (III), (III′), or (III″) or a salt or physiologically functional derivative thereof) and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. Most preferably the solvent used is water.

As used herein, the term “substituted” refers to substitution with the named substituent or substituents, multiple degrees of substitution being allowed unless otherwise stated.

Certain of the compounds described herein may contain one or more chiral atoms, or may otherwise be capable of existing as two enantiomers. The compounds of this invention include mixtures of enantiomers as well as purified enantiomers or enantiomerically enriched mixtures. Also included within the scope of the invention are the individual isomers of the compounds represented by formulae formulae (I), (II), (III), (III′), or (III″) as well as any wholly or partially equilibrated mixtures thereof. The present invention also covers the individual isomers of the compounds represented by the formulas above as mixtures with isomers thereof in which one or more chiral centers are inverted. Also, it is understood that any tautomers and mixtures of tautomers of the compounds of formulae (I), (II), (III), III′), or (III″) are included within the scope of the compounds of formulae (I), (II), (III), (III′), or (III″).

In one embodiment a method of treating cancer is provided which includes administering a therapeutically effective amount of at least one erb family inhibitor and at least one IGF-IR inhibitor.

In one embodiment, the erb family inhibitor is a dual inhibitor of erbB-2 and EGFR. Generally, any EGFR/erbB-2 inhibitor, that is, any pharmaceutical agent having specific erbB-2 and/or EGFR inhibitor activity may be utilized in the present invention. Such erbB-2/EGFR inhibitors are described, for instance, in U.S. Pat. Nos. 5,773,476; 5,789,427; 6,103,728; 6,169,091; 6,174,889; and 6,207,669; and International Patent Applications WO 95/24190; WO 98/0234; WO 99/35146; WO 01/04111; and WO 02/02552 which patents and patent applications are herein incorporated by reference to the extent of their disclosure of erbB-2 and/or EGFR inhibitor compounds as well as methods of making the same.

In one embodiment of the present invention, the dual EGFR/erbB-2 inhibitor compounds are of the Formula I:

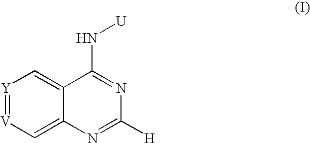

or a salt or solvate thereof;
wherein

### Y is CR¹ and V is N;

### or Y is CR¹ and V is CR²;

R¹ represents a group CH₃SO₂CH₂CH₂NHCH₂—Ar—, wherein Ar is selected from phenyl, furan, thiophene, pyrrole and thiazole, each of which may optionally be substituted by one or two halo, C₁₋₄ alkyl or C₁₋₄ alkoxy groups;
R² is selected from the group comprising hydrogen, halo, hydroxy, C₁₋₄ alkyl, C₁₋₄ alkoxy, C₁₋₄ alkylamino and di[C₁₋₄ alkyl]amino;
U represents a phenyl, pyridyl, 3H-imidazolyl, indolyl, isoindolyl, indolinyl, isoindolinyl, 1H-indazolyl, 2,3-dihydro-1H-indazolyl, 1H-benzimidazolyl, 2,3-dihydro-1H-benzimidazolyl or 1H-benzotriazolyl group, substituted by an R³ group and optionally substituted by at least one independently selected R⁴ group;
R³ is selected from a group comprising benzyl, halo-, dihalo- and trihalobenzyl, benzoyl, pyridylmethyl, pyridylmethoxy, phenoxy, benzyloxy, halo-, dihalo- and trihalobenzyloxy and benzenesulphonyl;
or R³ represents trihalomethylbenzyl or trihalomethylbenzyloxy;
or R³ represents a group of formula

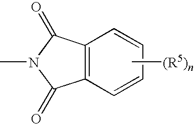

wherein each R⁵ is independently selected from halogen, C₁₋₄ alkyl and C₁₋₄ alkoxy;
and n is 0 to 3; and
each R⁴ is independently hydroxy, halogen, C₁₋₄ alkyl, C₂₋₄ alkenyl, C₂₋₄ alkynyl, C₁₋₄ alkoxy, amino, C₁₋₄ alkylamino, di[C₁₋₄ alkyl]amino, C₁₋₄ alkylthio, C₁₋₄ alkylsulphinyl, C₁₋₄ alkylsulphonyl, C₁₋₄ alkylcarbonyl, carboxy, carbamoyl, C₁₋₄ alkoxycarbonyl, C₁₋₄ alkanoylamino, N—(C₁₋₄ alkyl)carbamoyl, N,N-di(C₁₋₄ alkyl)carbamoyl, cyano, nitro and trifluoromethyl.

The definitions for Y and V thus give rise to two possible basic ring systems for the compounds of formula (I). In particular the compounds may contain the following basic ring systems: quinazolines (1) and pyrido-pyrimidines (2):

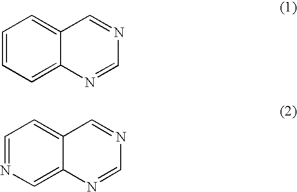

In one embodiment, the ring system is ring (1).

Suitable values for the various groups listed above within the definitions for R¹, R², R⁴ and R⁵ are as follows:

halo is, for example, fluoro, chloro, bromo or iodo; in one embodiment it is fluoro, chloro or bromo, in another embodiment it is fluoro or chloro;
C₁₋₄ alkyl is, for example, methyl, ethyl, propyl, isopropyl, butyl, isobutyl, sec-butyl or tert-butyl; in one embodiment it is methyl, ethyl, propyl, isopropyl or butyl, in another embodiment methyl;
C₂₋₄ alkenyl is, for example, ethenyl, prop-1-enyl or prop-2-enyl; in one embodiment ethenyl;
C₂₋₄ alkynyl is, for example, ethynyl, prop-1-ynyl or prop-2-ynyl; in one embodiment ethynyl;
C₁₋₄ alkoxy is, for example, methoxy, ethoxy, n-propoxy, isopropoxy, n-butoxy, isobutoxy, sec-butoxy or tert-butoxy; in one embodiment methoxy, ethoxy, propoxy, isopropoxy or butoxy; in another embodiment methoxy;
C₁₋₄ alkylamino is, for example, methylamino, ethylamino or propylamino; in one embodiment methylamino;
di[C₁₋₄ alkyl]amino is, for example, dimethylamino, diethylamino, N-methyl-N-ethylamino or dipropylamino; in one embodiment dimethylamino;
C₁₋₄ alkylthio is, for example, methylthio, ethylthio, propylthio or isopropylthio, in one embodiment methylthio;
C₁₋₄ alkylsulphinyl is, for example, methylsulphinyl, ethylsulphinyl, propylsulphinyl or isopropylsulphinyl, in one embodiment methylsulphinyl;
C₁₋₄ alkylsulphonyl is, for example, methanesulphonyl, ethylsulphonyl, propylsulphonyl or isopropylsulphonyl, in one embodiment methanesulphonyl;
C₁₋₄ alkylcarbonyl is, for example methylcarbonyl, ethylcarbonyl or propylcarbonyl;
C₁₋₄ alkoxycarbonyl is, for example, methoxycarbonyl, ethoxycarbonyl, propoxycarbonyl, butoxycarbonyl or tert-butoxycarbonyl;
C₁₋₄ alkanoylamino (where the number of carbon atoms includes the CO functionality) is, for example, formamido, acetamido, propionamido or butyramido;
N—(C₁₋₄ alkyl)carbamoyl is, for example, N-methylcarbamoyl or N-ethylcarbamoyl; and
N,N-di(C₁₋₄ alkyl)carbamoyl is, for example, N,N-dimethylcarbamoyl, N-methyl-N-ethylcarbamoyl or N,N-diethylcarbamoyl.

In one embodiment, Y is CR¹ and V is CR² (ring system (1) above).

In another embodiment, Y is CR¹ and V is N (ring system (2) above).

In one embodiment, R² represents hydrogen or C₁₋₄ alkoxy.

In another embodiment, R² represents hydrogen or methoxy.

In still another embodiment, R² represents halo; in one embodiment R² is fluoro.

In one embodiment, the group Ar is substituted by one halo, C₁₋₄ alkyl or C₁₋₄ alkoxy group.

In another embodiment, the group Ar is substituted by a C₁₋₄ alkyl group.

In still another embodiment, the group Ar does not carry any optional substituents.

In a further embodiment, Ar represents furan, phenyl or thiazole, each of which may optionally be substituted as indicated above.

In another embodiment, Ar represents furan or thiazole, each of which may optionally be substituted as indicated above.

In still another embodiment, Ar represents unsubstituted furan or thiazole.

The side chain CH₃SO₂CH₂CH₂NHCH₂ may be linked to any suitable position of the group Ar. Similarly, the group R¹ may be linked to the carbon atom carrying it from any suitable position of the group Ar.

In one embodiment, when Ar represents furan the side chain CH₃SO₂CH₂CH₂NHCH₂ is in the 4-position of the furan ring and the link to the carbon atom carrying the group R¹ is from the 2-position of the furan ring.

In another embodiment, when Ar represents furan the side chain CH₃SO₂CH₂CH₂NHCH₂ is in the 3-position of the furan ring and the link to the carbon atom carrying the group R¹ is from the 2-position of the furan ring.

In still another embodiment, when Ar represents furan the side chain CH₃SO₂CH₂CH₂NHCH₂ is in the 5-position of the furan ring and the link to the carbon atom carrying the group R¹ is from the 2-position of the furan ring.

In a further embodiment, when Ar represents thiazole the side chain CH₃SO₂CH₂CH₂NHCH₂ is in the 2-position of the thiazole ring and the link to the carbon atom carrying the group R¹ is from the 4-position of the thiazole ring.

The R³ and R⁴ groups may be bound to the ring system U by either a carbon atom or a heteroatom of the ring system. The ring system itself may be bound to the bridging NH group by a carbon atom or a heteroatom but is preferably bound by a carbon atom. The R³ and R⁴ groups may be bound to either ring when U represents a bicyclic ring system, but these groups are preferably bound to the ring which is not bound to the bridging NH group in such a case.

In one embodiment U, represents a phenyl, indolyl, or 1H-indazolyl group substituted by an R³ group and optionally substituted by at least one independently selected R⁴ group.

In another embodiment, U represents a phenyl or 1H-indazolyl group substituted by an R³ group and optionally substituted by at least one independently selected R⁴ group.

In still another embodiment, where U represents a phenyl group the group R³ is in the para-position relative to the bond from U to the linking NH group.

In a further embodiment, where U represents a 1H-indazolyl group the group R³ is in the 1-position of the indazolyl group.

In one embodiment, R³ represents benzyl, pyridylmethyl, phenoxy, benzyloxy, halo-, dihalo- and trihalobenzyloxy and benzenesulphonyl.

In another embodiment, R³ represents trihalomethylbenzyloxy.

In still another embodiment, R³ represents a group of formula

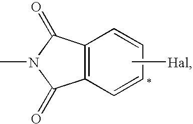

wherein Hal is Br or Cl, in one embodiment Cl, in another embodiment the Hal substituent is in the position marked with a star in the ring as shown.

In one embodiment, R³ represents benzyloxy, fluorobenzyloxy (especially 3-fluorobenzyloxy), benzyl, phenoxy and benzenesulphonyl.

In another embodiment R³ represents bromobenzyloxy (especially 3-bromobenzyloxy) and trifluoromethylbenzyloxy.

In still another embodiment, the ring U is not substituted by an R⁴ group; in an especially preferred embodiment U is phenyl or indazolyl unsubstituted by an R⁴ group.

In another embodiment, the ring U is substituted by an R⁴ group selected from halo or C₁₋₄ alkoxy; in one embodiment chloro, fluoro or methoxy.

In another embodiment, the ring U is substituted by an R⁴ group wherein R⁴ represents halo, in one embodiment 3-fluoro.

In one embodiment, U together with R⁴ represents methoxyphenyl, fluorophenyl, trifluoromethylphenyl or chlorophenyl.

In another embodiment, U together with R⁴ represents methoxyphenyl or fluorophenyl.

In another embodiment, the group U together with the substituent(s) R³ and

R⁴ represents benzyloxyphenyl, (fluorobenzyloxy)phenyl, (benzenesulphonyl)phenyl, benzylindazolyl or phenoxyphenyl.

In still another preferred embodiment, the group U together with the substituent(s) R³ and R⁴ represents benzyloxyphenyl, (3-fluorobenzyloxy)phenyl, (benzenesulphonyl)phenyl or benzylindazolyl.

In a further embodiment, the group U together with the substituent(s) R³ and

R⁴ represents (3-bromobenzyloxy)phenyl, (3-trifluoromethylbenzyloxy)phenyl, or (3-fluorobenzyloxy)-3-methoxyphenyl.

In another embodiment, the group U together with the substituent(s) R³ and R⁴ represents 3-fluorobenzyloxy-3-chlorophenyl, benzyloxy-3-chlorophenyl, benzyloxy-3-trifluoromethylphenyl, (benzyloxy)-3-fluorophenyl, (3-fluorobenzyloxy)-3-fluorophenyl or (3-fluorobenzyl)indazolyl.

In one embodiment, the group U together with the substituent(s) R³ and R⁴ represents benzyloxyphenyl or (3-fluorobenzyloxy)phenyl.

In another embodiment, there is provided a compound of formula (I) or a salt, or solvate thereof wherein V is CR², wherein R² is hydrogen, halo (in one embodiment fluoro) or C₁₋₄ alkoxy (in one embodiment methoxy); Y is CR¹ wherein R¹ is as defined above in which Ar is unsubstituted phenyl, furan or thiazole; U is phenyl or indazole; R³ is benzyl, fluorobenzyl, benzyloxy, fluorobenzyloxy, bromobenzyloxy, trifluoromethylbenzyloxy, phenoxy or benzenesulphonyl; and R⁴ is not present or is halo (in one embodiment chloro or fluoro), or methoxy.

In another embodiment, there is provided a compound of formula (I) or a salt or solvate thereof wherein V is CR², wherein R² is hydrogen, halo (in one embodiment fluoro) or C₁₋₄ alkoxy (in one embodiment methoxy); Y is CR¹ wherein R¹ is as defined above in which Ar is unsubstituted furan or thiazole; U is phenyl; R³ is benzyloxy, fluorobenzyloxy or benzenesulphonyl; and R⁴ is not present or is halo (in one embodiment chloro or fluoro), or methoxy.

In one embodiment, there is provided a compound of formula (I) or a salt or solvate thereof wherein V is CR², wherein R² is hydrogen, halo (in one embodiment fluoro) or C₁₋₄ alkoxy (in one embodiment methoxy); Y is CR¹ wherein R¹ is as defined above in which Ar is unsubstituted furan or thiazole; U is indazole; R³ is benzyl or fluorobenzyl; and R⁴ is not present.

In another embodiment, there is provided a compound of formula (I) or a salt or solvate thereof wherein Y is CR², wherein R² is hydrogen, halo (in one embodiment fluoro) or C₁₋₄ alkoxy (in one embodiment methoxy); V is CR¹ wherein R¹ is as defined above in which Ar is unsubstituted phenyl, furan or thiazole; U is phenyl or indazole; R³ is benzyl, fluorobenzyl, benzyloxy, fluorobenzyloxy, bromobenzyloxy, trifluoromethylbenzyloxy, phenoxy or benzenesulphonyl; and R⁴ is not present or is halo (in one embodiment chloro or fluoro), or methoxy.

In another embodiment, there is provided a compound of formula (I) or a salt or solvate thereof wherein Y is CR², wherein R² is hydrogen, halo (in one embodiment fluoro) or C₁₋₄ alkoxy (in one embodiment methoxy); V is CR¹ wherein R¹ is as defined above in which Ar is unsubstituted furan or thiazole; U is phenyl; R³ is benzyloxy, fluorobenzyloxy or benzenesulphonyl; and R⁴ is not present or is halo (in one embodiment chloro or fluoro), or methoxy.

In another embodiment, there is provided a compound of formula (I) or a salt or solvate thereof wherein Y is CR², wherein R² is hydrogen, halo (in one embodiment fluoro) or C₁₋₄ alkoxy (in one embodiment methoxy); V is CR¹ wherein R¹ is as defined above in which Ar is unsubstituted furan or thiazole; U is indazole; R³ is benzyl or fluorobenzyl; and R⁴ is not present.

In another embodiment, there is provided a compound of formula (I) or a salt or solvate thereof wherein Y is CR², wherein R² is hydrogen, halo (in one embodiment fluoro) or C₁₋₄ alkoxy (in one embodiment methoxy); V is CR¹ wherein R¹ is as defined above in which Ar is unsubstituted furan or thiazole; U is phenyl; R³ is phenoxy; and R⁴ is not present.

In another embodiment, there is provided a compound of formula (I) or a salt or solvate thereof wherein V is N; Y is CR¹ wherein R¹ is as defined above in which Ar is unsubstituted phenyl, furan or thiazole; U is phenyl or indazole; R³ is benzyl, fluorobenzyl, benzyloxy, fluorobenzyloxy, bromobenzyloxy, trifluoromethylbenzyloxy, phenoxy or benzenesulphonyl; and R⁴ is not present or is halo (in one embodiment chloro or fluoro), or methoxy.

In another embodiment, there is provided a compound of formula (I) or a salt or solvate thereof wherein V is N, Y is CR¹ wherein R¹ is as defined above in which Ar is unsubstituted furan or thiazole; U is phenyl; R³ is benzyloxy, fluorobenzyloxy or benzenesulphonyl; and R⁴ is not present or is halo (in one embodiment chloro or fluoro), or methoxy.

In another embodiment, there is provided a compound of formula (I) or a salt or solvate thereof wherein V is N, Y is CR¹ wherein R¹ is as defined above in which Ar is unsubstituted furan or thiazole; U is indazole; R³ is benzyl or fluorobenzyl; and R⁴ is not present.

In another embodiment, the compound of formula (I) is a compound of formula (II):

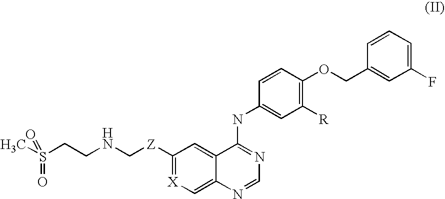

or a salt or solvate thereof, wherein R is —Cl or —Br, X is CH, N, or CF, and Z is thiazole or furan.

In another embodiment, the compound of formula (I) is a compound of formula (III):

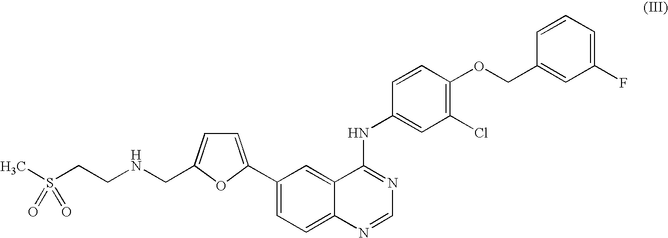

or a salt or solvate thereof.

In another embodiment, the compound of formula (I) is a ditosylate salt of the compound of formula (III) or anhydrate or hydrate forms thereof. The ditosylate salt of the compound of formula (III) has the chemical name N-{3-chloro-4-[(3-fluorobenzyl) oxy]phenyl}-6-[5-({[2-(methanesulphonyl)ethyl]amino}methyl)-2-furyl]-4-quinazolinamine ditosylate. In one embodiment, the compound of formula (I) is the anhydrous ditosylate salt of the compound of formula (III). In another embodiment, the compound of formula (I) is the monohydrate ditosylate salt of the compound of formula (III).

In another embodiment, the compound of formula (I) is a compound of formula (II) wherein, R is Cl; X is CH; and Z is thiazole. In another embodiment, the compound of formula (I) is a ditosylate salt of a compound of formula (II) wherein, R is Cl; X is CH; and Z is thiazole; or anhydrate or hydrate forms thereof. The chemical name of such compound of formula (II) is (4-(3-fluoro-benzyloxy)-3-chlorophenyl)-(6-(2-((2-methanesulphonyl-ethylamino)-methyl)-thiazol-4-yl)quinazolin-4-yl)-amine and is a compound of formula (III′).

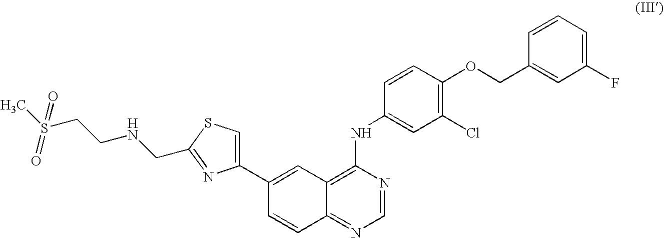

In another embodiment, the compound of formula (I) is a compound of formula (II) wherein, R is Br; X is CH; and Z is furan. In another embodiment, the compound of formula (I) is a ditosylate salt of the compound of formula (II) wherein, R is Br; X is CH; and Z is furan; or anhydrate or hydrate forms thereof. The chemical name of such compound of formula (II) is (4-(3-fluoro-benzyloxy)-3-bromophenyl)-(6-(5-((2-methanesulphonyl-ethylamino)-methyl)-furan-2-yl)quinazolin-4-yl)-amine and is a compound of formula (III″).

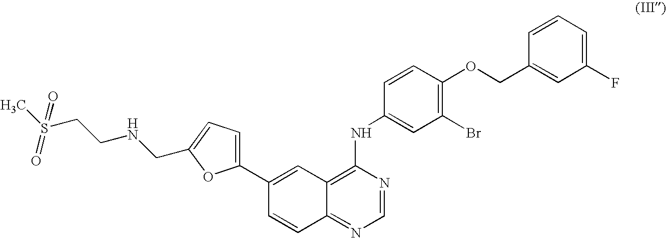

The free base, HCl salts, and ditosylate salts of the compounds of Formulae (I), (II), (III), (III′) and (III″) may be prepared according to the procedures of International Patent Application No. PCT/EP99/00048, filed Jan. 8, 1999, and published as WO 99/35146 on Jul. 15, 1999, referred to above and International Patent Application No. PCT/US01/20706, filed Jun. 28, 2001 and published as WO 02/02552 on Jan. 10, 2002 and according to the appropriate Examples recited below. One such procedure for preparing the ditosylate salt of the compound of formula (III) is presented following in Scheme A

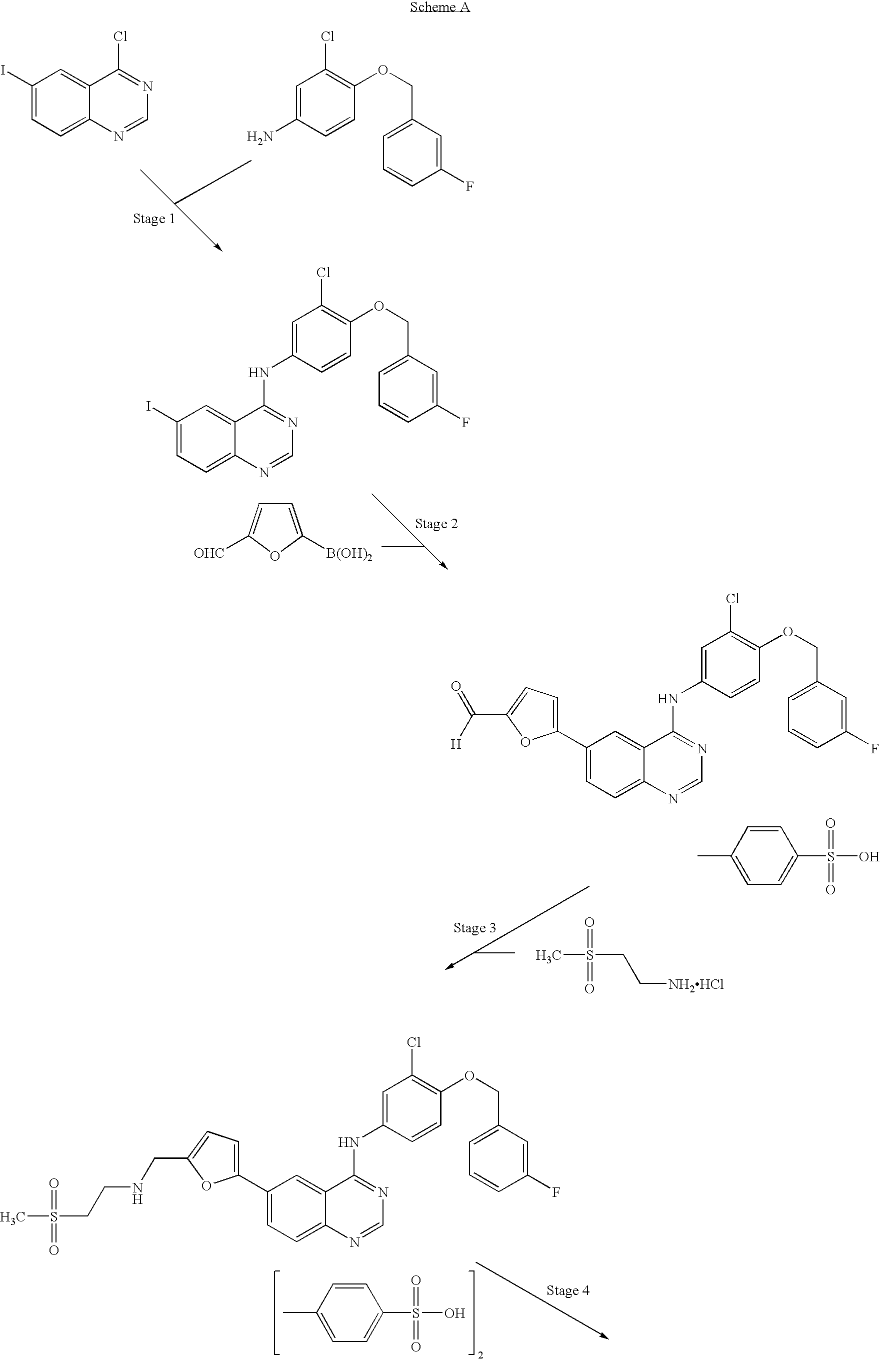

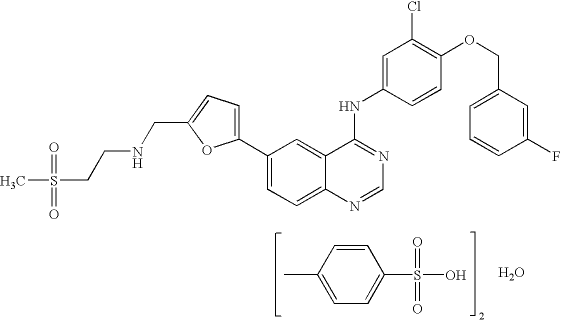

In scheme 1, the preparation of the ditosylate salt of the compound of formula (III) proceeds in four stages: Stage 1: Reaction of the indicated bicyclic compound and amine to give the indicated iodoquinazoline derivative; Stage 2: preparation of the corresponding aldehyde salt; Stage 3: preparation of the quinazoline ditosylate salt; and Stage 4: monohydrate ditosylate salt preparation.

Scheme B following illustrates the preparation of the ditosylate salt of the compound of formula (II). The preparation proceeds in four stages: Stage 1: reaction of quinazoline (I), which is prepared from 3H-6-iodoquinazolin-4-one (I′), with amine (II) to give iodoquinazoline (III); Stage 2: preparation of the corresponding aldehyde salt (V) by reaction of iodoquinazoline (III) and boronic acid (IV) followed by treatment with p-toluenesulfonic acid salt; Stage 3: preparation of the ditosylate salt of GW572016 (VI) from aldehyde salt (V); and Stage 4: recrystallization of the GW572016 ditosylate salt (VI). Scheme C shows an alternate preparation of the ditosylate salt of the compound of formula (III).

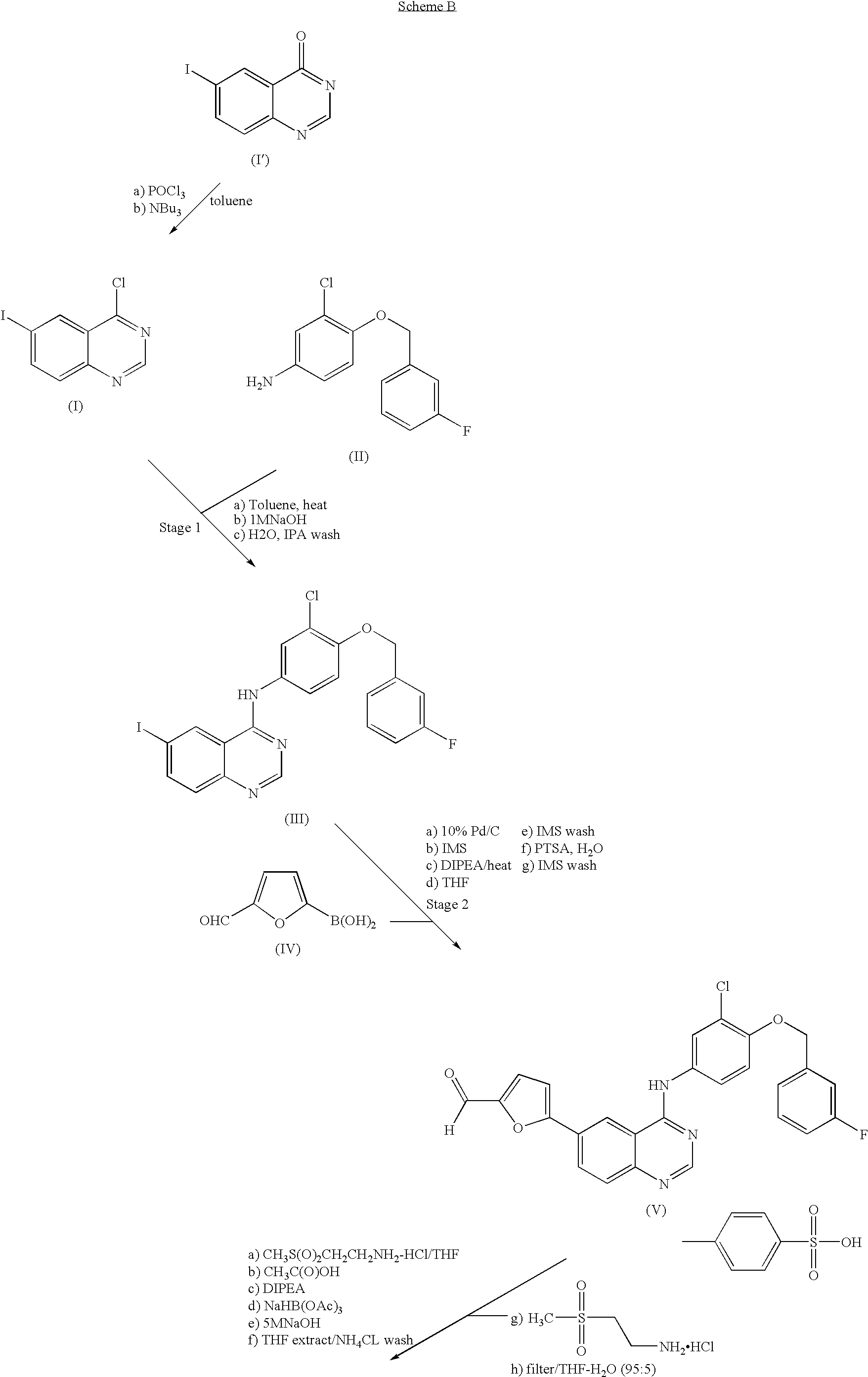

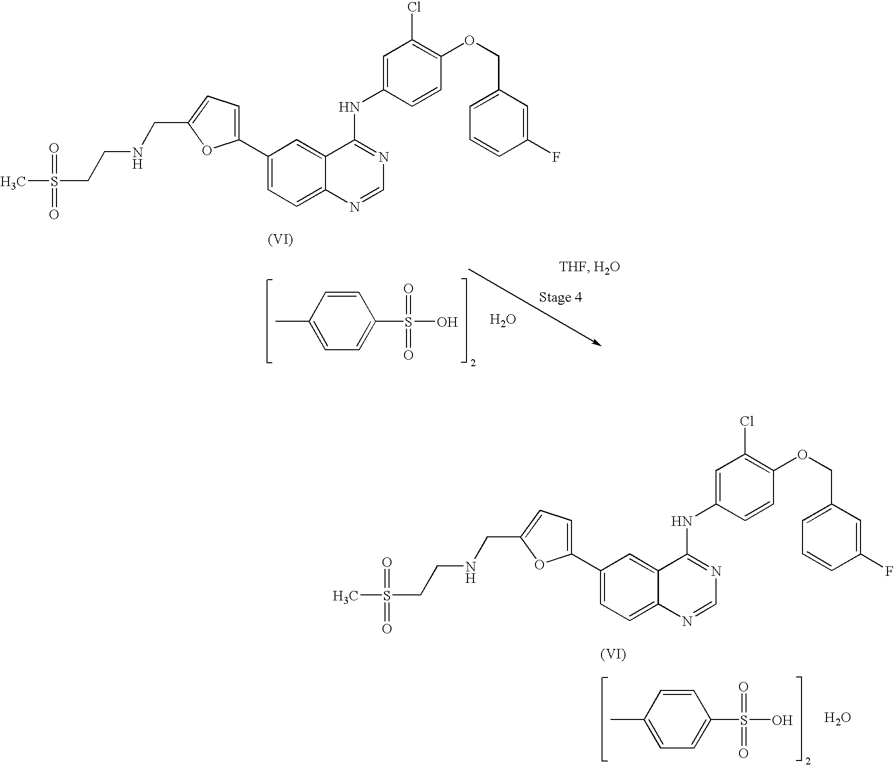

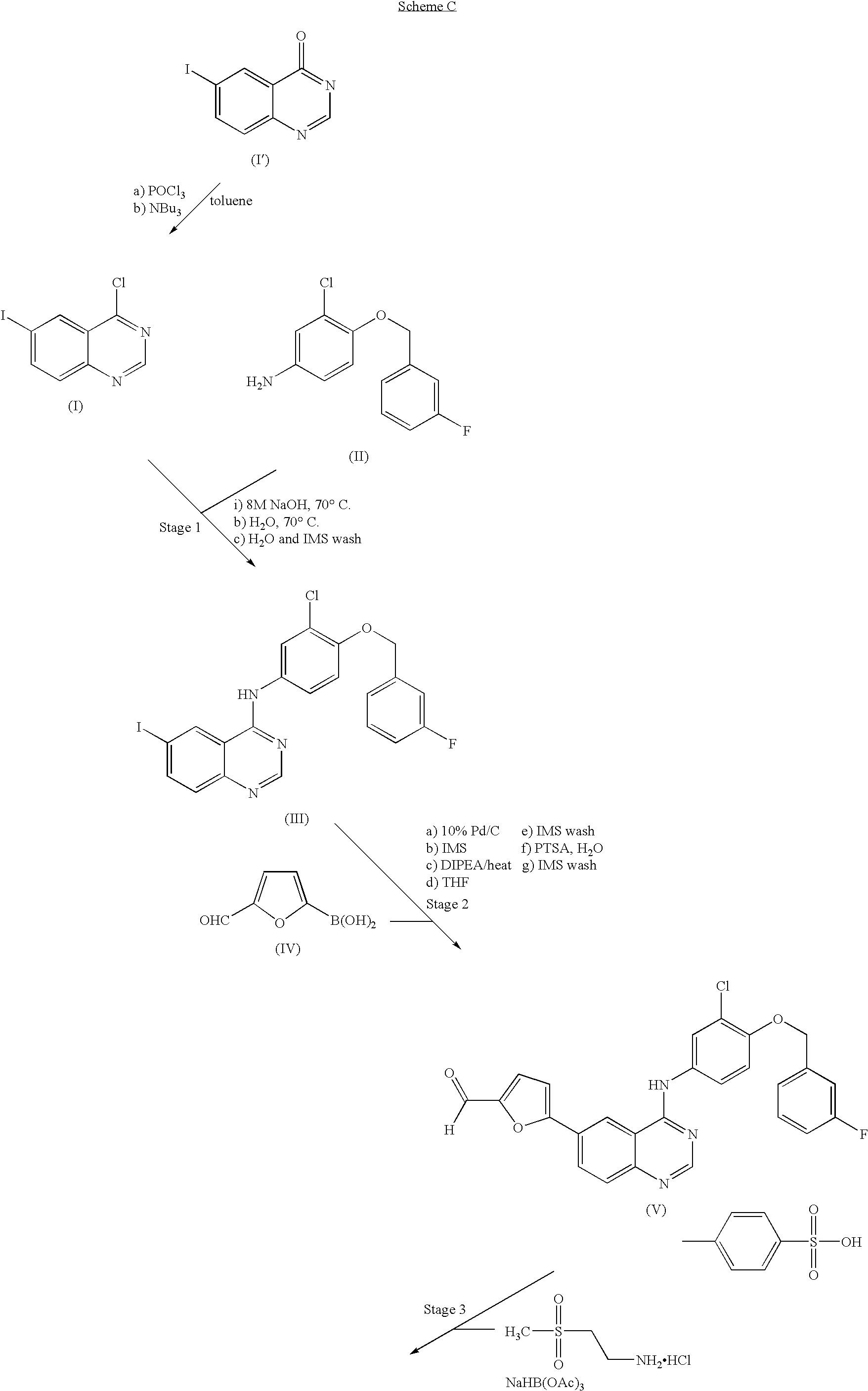

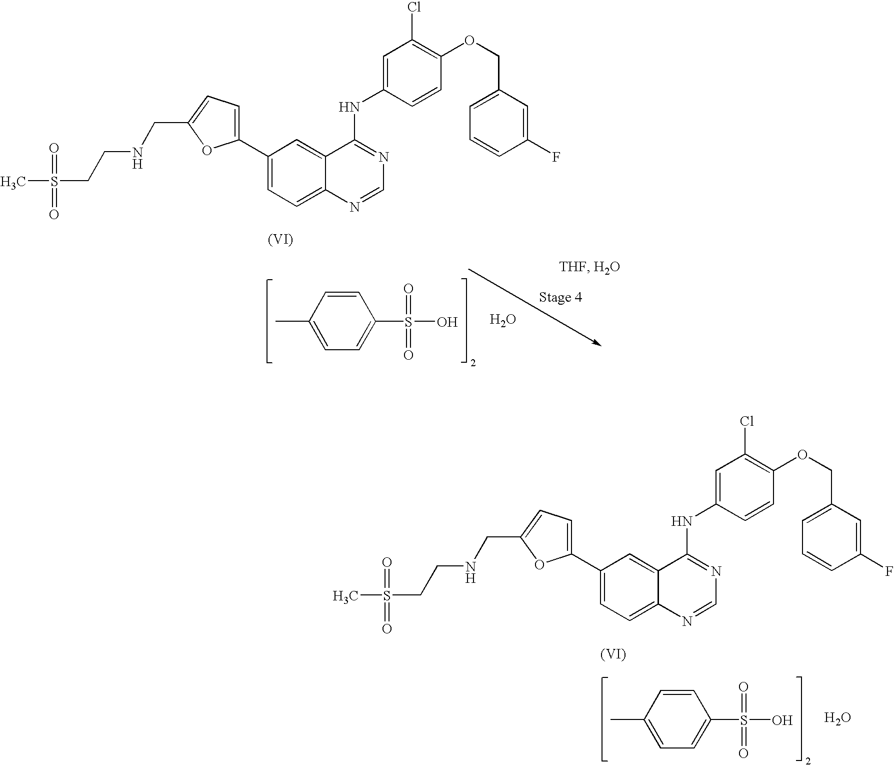

As recited above the method and treatment combination of the present invention also includes at least one IGF-1R inhibitor. Generally any IGF-1R inhibitor, that is, any pharmaceutical agent having specific IGF-1R inhibitor activity may be utilized in the present invention. Such IGF-1R inhibitors may be small molecule IGF-1R inhibitors, antisense oligonucleotides to IGF-1R, peptides inhibitors of IGF-1R; or fully human, monoclonal, or recombinant antibodies to IGF-1R. Suitable examples include the development compounds INSM-18 from Insmed; BMS-577098 from Bristol-Myers Squibb; INX 4437 of Inex; SB144, YM 17, YM 27, SSP 1, and SSP 5 of STIL Biotechnologies, DAX-21834 of Telik; IMC-A12 of Imclone, EM-164 of ImmunoGen; 19D12 of Medarex; and F-50035 of Pierre Fabre.

Suitable examples also include those IGF-1R inhibitors, including NVP-ADW-742 or NVP-AEW541, described in PCT Application PCT/EP02/05239 filed May 13, 2002 and published as International Patent Application WO 02/092599 on Nov. 11, 2002; those disclosed in U.S. Pat. No. 6,337,338 issued Jan. 8, 2002; and in U.S. Pat. Nos. 6,340,674; 6,506,415; 6,541,036; and 6,596,473.

The erb family inhibitor, e.g., dual EGFR/erbB-2 inhibitor and the IGF-1R inhibitor, may be employed in combination in accordance with the invention by administration concomitantly in (1) a unitary pharmaceutical composition including both compounds or (2) separate pharmaceutical compositions each including one of the compounds. Alternatively, the combination may be administered separately in a sequential manner wherein, for example, the IGF-1R inhibitor or dual EGFR/erbB-2 inhibitor is administered first and the other second. Such sequential administration may be close in time or remote in time.

Typically, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts derived from a nitrogen on a substituent in a compound of the present invention. Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, tannate, tartrate, teoclate, tosylate, triethiodide, trimethylammonium and valerate. Other salts, which are not pharmaceutically acceptable, may be useful in the preparation of compounds of this invention and these form a further aspect of the invention.

While it is possible that, for use in therapy, therapeutically effective amounts of a dual EGFR/erbB2 and IGF-1R inhibitor, as well as salts or solvates thereof, may be administered as the raw chemical, it is possible to present the active ingredient as a pharmaceutical composition. Accordingly, the invention further provides pharmaceutical compositions, which include therapeutically effective amounts of a dual EGFR/erbB2 and/or IGF-1R inhibitor and salts or solvates thereof, and one or more pharmaceutically acceptable carriers, diluents, or excipients. The compounds of the present invention and salts or solvates thereof, are as described above. The carrier(s), diluent(s) or excipient(s) must be acceptable in the sense of being compatible with the other ingredients of the formulation and not deleterious to the recipient thereof. In accordance with another aspect of the invention there is also provided a process for the preparation of a pharmaceutical formulation including admixing a dual EGFR/erbB2 and/or a IGF-1R inhibitor or salts or solvates thereof, with one or more pharmaceutically acceptable carriers, diluents or excipients.

Pharmaceutical formulations may be presented in unit dose forms containing a predetermined amount of active ingredient per unit dose. Such a unit may contain, for example, 0.5 mg to 1 g, 1 mg to 700 mg, or 5 mg to 100 mg of an EGFR/erbB2 and/or IGF-1R inhibitor, depending on the condition being treated, the route of administration and the age, weight and condition of the patient, or pharmaceutical formulations may be presented in unit dose forms containing a predetermined amount of active ingredient per unit dose. Preferred unit dosage formulations are those containing a daily dose or sub-dose, as herein above recited, or an appropriate fraction thereof, of an active ingredient. Furthermore, such pharmaceutical formulations may be prepared by any of the methods well known in the pharmacy art.

The dual EGFR/erbB-2 inhibitors and IGF-1R inhibitors may be administered by any appropriate route. Suitable routes include oral, rectal, nasal, topical (including buccal and sublingual), vaginal, and parenteral (including subcutaneous, intramuscular, intraveneous, intradermal, intrathecal, and epidural). It will be appreciated that the preferred route may vary with, for example, the condition of the recipient of the combination. It will also be appreciated that each of the agents administered may be administered by the same or different routes and that the erbB-2 and IGF-1R inhibitors may be compounded together in a pharmaceutical composition/formulation.

The method of the present invention may also be employed with other therapeutic methods of cancer treatment. In particular, in anti-neoplastic therapy, combination therapy with other chemotherapeutic, hormonal, antibody agents as well as surgical and/or radiation treatments other than those mentioned above are envisaged. Anti-neoplastic therapies are described for instance in International Application No. PCT US 02/01130, filed Jan. 14, 2002, which application is incorporated by reference to the extent that it discloses anti-neoplastic therapies. Combination therapies according to the present invention thus include the administration of at least one erbB-2 inhibitor and at least one IGF-1R inhibitor as well as optional use of other therapeutic agents including other anti-neoplastic agents. Such combination of agents may be administered together or separately and, when administered separately this may occur simultaneously or sequentially in any order, both close and remote in time. The amounts of the erbB2 and IGF-1R inhibitors and the other pharmaceutically active agent(s) and the relative timings of administration will be selected in order to achieve the desired combined therapeutic effect.

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also to granulating, the powder mixture can be run through the tablet machine and the result is imperfectly formed slugs broken into granules. The granules can be lubricated be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to prevent sticking to the tablet forming dies by means of the addition of stearic acid, a stearate salt, talc or mineral oil. The lubricated mixture is then compressed into tablets. The compounds of the present invention can also be combined with free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax or the like.

The agents for use according to the present invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Agents for use according to the present invention may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986).

Pharmaceutical formulations adapted for topical administration may be formulated as ointments, creams, suspensions, lotions, powders, solutions, pastes, gels, sprays, aerosols or oils.

For treatments of the eye or other external tissues, for example mouth and skin, the formulations are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles and mouth washes.

Pharmaceutical formulations adapted for rectal administration may be presented as suppositories or as enemas.

Pharmaceutical formulations adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists that may be generated by means of various types of metered dose pressurised aerosols, nebulizers or insufflators.

Pharmaceutical formulations adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the formulations may include other agents conventional in the art having regard to the type of formulation in question, for example those suitable for oral administration may include flavoring agents.

Also, contemplated in the present invention is a pharmaceutical combination including at least one erb family inhibitor, such as a dual erbB-2/EGFR inhibitor and at least one IGF-1R inhibitor. In another embodiment, the pharmaceutical combination includes an erbB-2 inhibitor and an IGF-1R inhibitor, and optionally at least one additional anti-neoplastic agent. The erb inhibitors, IGF-1R inhibitors, and additional anti-neoplastic therapy are as described above.

As indicated, therapeutically effective amounts of the specific erb family inhibitor and IGF-1R inhibitor are administered to a mammal. Typically, the therapeutically effective amount of one of the administered agents of the present invention will depend upon a number of factors including, for example, the age and weight of the mammal, the precise condition requiring treatment, the severity of the condition, the nature of the formulation, and the route of administration. Ultimately, the therapeutically effective amount will be at the discretion of the attendant physician or veterinarian.

Typically, the erb family and IGF-1R inhibitors will be given in the range of 0.1 to 100 mg/kg body weight of recipient (mammal) per day and more usually in the range of 1 to 10 mg/kg body weight per day.

As indicated, the method of cancer treatment of the present invention, is directed to any susceptible cancer. Typically, the cancer is any cancer which is susceptible to inhibition of EGFR, erbB-2, and/or IGF-1R. Examples of cancers that are suitable for treatment by the method and treatment combination of the present invention include, but are not limited to, head and neck, breast, lung, colon, ovary, pancreatic, and prostate cancers.

The following examples are intended for illustration only and are not intended to limit the scope of the invention in any way.

### EXAMPLES

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:

| | |
|---|---|
| g (grams); | mg (milligrams); |
| L (liters); | mL (milliliters); |
| μL (microliters); | psi (pounds per square inch); |
| M (molar); | mM (millimolar); |
| N (Normal) | Kg (kilogram) |
| i.v. (intravenous); | Hz (Hertz); |
| MHz (megahertz); | mol (moles); |
| mmol (millimoles); | RT (room temperature); |
| min (minutes); | h (hours); |
| mp (melting point); | TLC (thin layer chromatography); |
| Tᵣ (retention time); | RP (reverse phase); |
| DCM (dichloromethane); | DCE (dichloroethane); |
| DMF (N,N-dimethylformamide); | HOAc (acetic acid); |
| TMSE (2-(trimethylsilyl)ethyl); | TMS (trimethylsilyl); |
| TIPS (triisopropylsilyl); | TBS (t-butyldimethylsilyl); |

HPLC (high pressure liquid chromatography);

| | |
|---|---|
| THF (tetrahydrofuran); | DMSO (dimethylsulfoxide); |
| EtOAc (ethyl acetate); | DME (1,2-dimethoxyethane); |

EDTA ethylenediaminetetraacetic acid
FBS fetal bovine serum
IMDM Iscove's Modified Dulbecco's medium
PBS phosphate buffered saline
RPMI Roswell Park Memorial Institute
RIPA buffer*
RT room temperature
*150 mM NaCl, 50 mM Tris-HCl, pH 7.5, 0.25% (w/v) -deoxycholate, 1% NP-40, 5 mM sodium orthovanadate, 2 mM sodium fluoride, and a protease inhibitor cocktail.

Unless otherwise indicated, all temperatures are expressed in ° C. (degrees Centigrade). All reactions conducted under an inert atmosphere at room temperature unless otherwise noted.

¹H NMR spectra were recorded on a Varian VXR-300, a Varian Unity-300, a Varian Unity-400 instrument, or a General Electric QE-300. Chemical shifts are expressed in parts per million (ppm, δ units). Coupling constants are in units of hertz (Hz). Splitting patterns describe apparent multiplicities and are designated as s (singlet), d (doublet), t (triplet), q (quartet), m (multiplet), br (broad).

Low-resolution mass spectra (MS) were recorded on a JOEL JMS-AX505HA, JOEL SX-102, or a SCIEX-APIiii spectrometer; high resolution MS were obtained using a JOEL SX-102A spectrometer. All mass spectra were taken under electrospray ionization (ESI), chemical ionization (CI), electron impact (EI) or by fast atom bombardment (FAB) methods. Infrared (IR) spectra were obtained on a Nicolet 510 FT-IR spectrometer using a 1-mm NaCl cell. All reactions were monitored by thin-layer chromatography on 0.25 mm E. Merck silica gel plates (60F-254), visualized with UV light, 5% ethanolic phosphomolybdic acid or p-anisaldehyde solution. Flash column chromatography was performed on silica gel (230-400 mesh, Merck). Optical rotations were obtained using a Perkin Elmer Model 241 Polarimeter. Melting points were determined using a MeI-Temp II apparatus and are uncorrected.

Examples 1-8 recite the preparation of specific erbB-2/EGFR inhibitors useful in the present invention.

### Example 1

### Monohydrate Ditosylate Salt of N-{3-Chloro-4-[(3-fluorobenzyl)oxy]phenyl}-6-[5-({[2-(methane sulphonyl)ethyl]amino}methyl)-2-furyl]-4-quinazolinamine (Monohydrate Ditosylate Salt of Compound of Formula (III))

### 1(a) Preparation of N-{3-Chloro-4-[(3-fluorobenzyl)oxy]phenyl}-6-[5-({[2-(methane sulphonyl)ethyl]amino}methyl)-2-furyl]-4-quinazolinamine (Free Base of Compound of Formula (III))

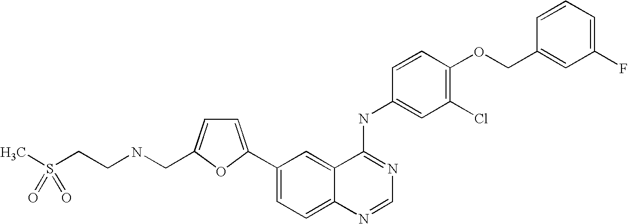

The title compound was prepared according to Procedure D of International Applications WO 02/02552: p. 16, line 19 to p. 17, line 3 and WO 99/35146: p. 56, lines 20-32 and Example 29p. 100, lines 18-29, from 5-(4-{3-chloro-4-(3-fluorobenzyloxy)-anilino}-6-quinazolinyl)-furan-2-carbaldehyde (0.6 equiv) and 2-methanesulphonyl-ethylamine (1 equiv). ¹H NMR 400 MHz (DMSO-d6) 9.60 (bs, 1H); 9.32 (bs, 1H); 8.82 (bs, 1H); 8.34 (d, 1H); 8.0 (s, 1H); 7.88 (d, 1H); 7.74 (d, 1H); 7.45 (m, 1H); 7.34-7.23 (m, 4H); 7.17 (m, 1H); 6.83 (d, 1H); 5.27 (s, 2H); 4.42 (s, 2H); 3.59 (m, 2H); 3.40 (m, 2H, obscured by waterpeak); 3.12 (s, 3H); MS m/z 581 (M+H⁺).

### 1(b) Preparation of Monohydrate Ditosylate Salt of N-{3-Chloro-4-[(3-fluorobenzyl)oxy]phenyl}-6-[5-({[2-(methane sulphonyl)ethyl]amino}methyl)-2-furyl]-4-quinazolinamine (Monohydrate Ditosylate Salt of Compound of Formula (III))

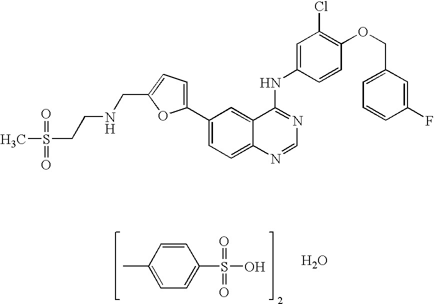

### Stage 1: Preparation of N-{3-chloro-4-[(3-fluorobenzyl)oxy]phenyl}-6-iodo-4-quinazolinamine

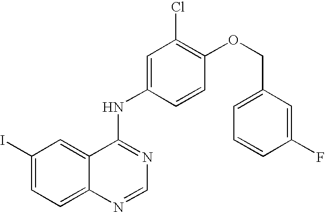

4-Chloro-6-iodoquinazoline (1 wt) was added to a solution of fluorobenzyloxyaniline (0.894 wt, 1.03 equiv) in N-methylpyrrolidinone (8.26 wt, 8 vol) at ca 20° C., and after the initial exotherm had subsided, the resulting solution was stirred at 20°-25° C. for at least 30 minutes. The dark solution was treated with triethylamine (0.58 vol, 1.2 equiv) and the mixture was stirred for 20-30 minutes. Isopropanol (2.5 vol) was added and the mixture was heated to ca 50° C. Water (up to 3 vol) was added slowly to the vessel over 10-15 minutes, while keeping the temperature at ca 50° C. Once crystallisation had commenced the addition was stopped and the resulting slurry was aged for 30-45 minutes at ca 50° C. Any residual water (from the 3 vol) was added, then further water (5 vol) was added to the vessel over ca 30 minutes while maintaining the temperature at ca 50° C. The resulting slurry was cooled to ca 20° C. over ca 30 minutes and aged at ca 20° C. for at least 30 minutes. The solid was collected by filtration and washed sequentially with water (2×5 vol), then isopropanol (5 vol). The product was dried in vacuo at ca 60° C. to give the title compound as a cream crystalline solid.

### Stage 2: Preparation of 5-(4-[3-chloro-4-(3-fluorobenzyloxy)-anilino]-6-quinazolinyl)-furan-2-carbaldehyde 4-methylbenzenesulfonate

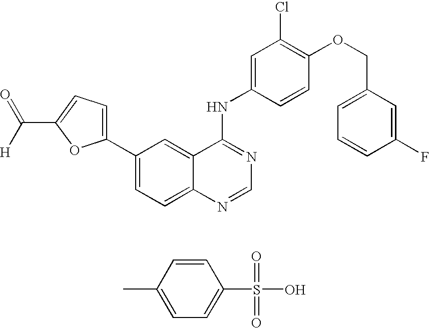

A mixture of N-{3-chloro-4-[(3-fluorobenzyl)oxy]phenyl}-6-iodo-4-quinazolinamine (1 wt), boronic acid (0.37 wt, 1.35 equiv), and 10% palladium on charcoal (0.028 wt 50% water wet) was slurried in IMS (15 vol). The resultant suspension was stirred for 5 minutes, treated with di-isopropylethylamine (0.39 vol, 1.15 equiv) and then heated to ca 70° C. for ca 3 hours when the reaction was complete (determined by HPLC analysis). The mixture was diluted with tetrahydrofuran (THF, 15 vol) and then filtered (hot—through GFA filter paper) to remove catalyst. The vessel was rinsed with IMS (2 vol).

A solution of p-toluenesulfonic acid monohydrate (1.54 wt, 4.1 equiv) in water (3 vol) was added over 5-10 minutes to the filtered solution maintained at 65° C. After crystallisation the suspension was stirred at 60°-65° C. for 1 hour, cooled to ca 25° C. over 1 hour and stirred at this temperature for a further 2 hours. The solid was collected by filtration, washed with IMS (3 vol) then dried in vacuo at ca 50° C. to give the tile compound as a yellow-orange crystalline solid.

### Stage 3: Preparation of Anhydrous Ditosylate Salt of N-{3-Chloro-4-[(3-fluorobenzyl)oxy]phenyl}-6-[5-({[2-(methane sulphonyl)ethyl]amino}methyl)-2-furyl]-4-quinazolinamine (Anhydrous Ditosylate Salt of Compound of Formula (III))

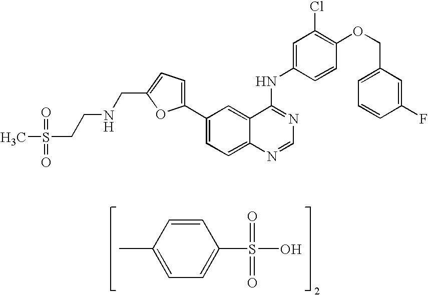

5-(4-[3-chloro-4-(3-fluorobenzyloxy)-anilino]-6-quinazolinyl)-furan-2-carbaldehyde 4-methylbenzenesulfonate (1 wt) and 2-(methylsulfonyl)ethylamine hydrochloride (0.4 wt, 1.6 equiv) were suspended in THF (10 vol). Sequentially, acetic acid (0.35 vol, 4 equiv) and di-isopropylethylamine (1.08 vol, 4 equiv) were added. The resulting solution was stirred at 30°-35° C. for ca 1 hour then cooled to ca 23° C. Sodium triacetoxyborohydride (0.66 wt, 2 equiv) was then added as a continual charge over approximately 15 minutes (some effervescence is seen at this point). The resulting mixture was stirred at ca 22° C. for ca 2 hours then sampled for HPLC analysis. The reaction was quenched by addition of 5M aqueous sodium hydroxide (5 vol) and stirred for ca 30 minutes (some effervescence is seen at the start of the caustic addition).

The aqueous phase was then separated, extracted with THF (2 vol) and the combined THF extracts were then washed with 10% w/v aqueous sodium chloride solution (4 vol). A solution of p-toluenesulfonic acid monohydrate (pTSA, 1.77 wt, 6 equiv) in THF (7 vol)¹ was prepared and warmed to ca 55° C. The THF solution of N-{3-Chloro-4-[(3-fluorobenzyl)oxy]phenyl}-6-[5-({[2-(methanesulphonyl)ethyl]amino}methyl)-2-furyl]-4-quinazolinamine was added to the pTSA solution over at least 30 minutes, maintaining the batch temperature at ca 55°±3° C. 2. The resulting suspension was stirred at ca 55° C. for 2 hours, cooled to 20°-25° C. over ca 60 minutes and aged at this temperature for ca 30 minutes. The solid was collected by filtration, washed with THF (2×2 vol) and dried in vacuo at ca 40° C. to give the desired compound as a pale yellow crystalline solid.

### Stage 4: Preparation of Monohydrate Ditosylate Salt of N-{3-Chloro-4-[(3-fluorobenzyl)oxy]phenyl}-6-[5-({[2-(methane sulphonyl)ethyl]amino}methyl)-2-furyl]-4-quinazolinamine (Monohydrate Ditosylate Salt of Compound of Formula (III))

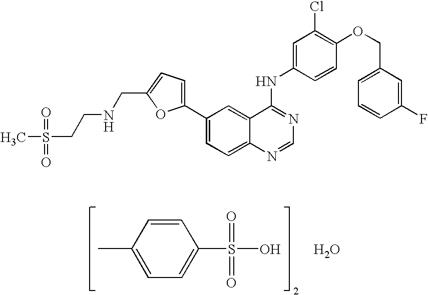

A suspension of the anhydrous ditosylate salt of N-{3-Chloro-4-[(3-fluorobenzyl)oxy]phenyl}-6-[5-({[2-(methane sulphonyl)ethyl]amino}methyl)-2-furyl]-4-quinazolinamine (1 wt), in tetrahydrofuran (THF, 14 vol) and water (6 vol) was heated to ca 55°-60° C. for 30 minutes to give a solution which was clarified by filtration and the lines washed into the crystallisation vessel with THF/Water (7:3, 2 vol). The resultant solution was heated to reflux and tetrahydrofuran (9 vol, 95% w/w azeotrope with water) was distilled off at atmospheric pressure.

The solution was seeded with N-{3-Chloro-4-[(3-fluorobenzyl)oxy]phenyl}-6-[5-({[2-(methane sulphonyl)ethyl]amino}methyl)-2-furyl]-4-quinazolinamine ditosylate monohydrate (0.002 wt). Once the crystallisation was established water (6 vol) was added while maintaining the reaction temperature above 55° C. The mixture was cooled to 5°-15° C. over ca 2 hours. The solid was collected by filtration, washed with tetrahydrofuran/water (3:7 ratio, 2×2 vol) and dried in vacuo at 45° C. to give N-{3-Chloro-4-[(3-fluorobenzyl)oxy]phenyl}-6-[5-({[2-(methane sulphonyl)ethyl]amino}methyl)-2-furyl]-4-quinazolinamine ditosylate monohydrate as a bright yellow crystalline solid.

### Example 2

### Preparation of N-{3-Chloro-4-[(3-fluorobenzyl)oxy]phenyl}-6-[5-({[2-(methanesulphonyl)ethyl]amino}methyl)-2-furyl]-4-quinazolin Amine Ditosylate Monohydrate

N-{3-Chloro-4-[(3-fluorobenzyl) oxy]phenyl}-6-[5-({[2-(methanesulphonyl)ethyl]amino}methyl)-2-furyl]-4-quinazolin amine ditosylate monohydrate may also be prepared according to the procedure of Scheme C as follows:

Stage 1—A stirred suspension of 3H-6-iodoquinazolin-4-one in toluene (5 vols) is treated with tri-n-butylamine (1.2 equiv.), and then heated to 70-80° C. Phosphorous oxychloride (1.1 equiv.) is added and the reaction mixture is then heated to reflux and stirred at this temperature for at least 2 hours. The reaction mixture is then cooled to 55° C. and toluene (5 vol) added followed by 3-chloro-4-{[(3-fluorophenyl)methyl]oxy}aniline (1.03 equiv.). The reaction mixture is then warmed to 70-90° C. and stirred for at least 2 hours. The resultant slurry is transferred to a second vessel. The temperature is adjusted to 70-75° C. and 8 molar aqueous sodium hydroxide solution (2 vols) added over 1 hour, followed by water (6 vol.) maintaining the contents at 70-85° C. The mixture is stirred at 70-85° C. for ca. 1 hour and then cooled to 20-25° C. The suspension is stirred for ca. 2 hours and the product collected by filtration, and washed successively with water, 0.1 molar aqueous sodium hydroxide, water, and IMS, then dried in vacuo.

Stage 2—A mixture of N-(3-chloro-4-{[(3-fluorophenyl)methyl]oxy}phenyl)-6-iodo-4-quinazolinamine (1 wt), (5-formyl-2-furanyl)boronic acid (0.374 wt, 1.35 eq) and 10% Palladium on charcoal (0.028 wt 50% water wet) is slurried in ethanol (industrial methylated spirits, 15 vols) to give a grey suspension. The resultant slurry is stirred for 5 minutes and then treated with N,N-di-isopropylethylamine (0.396 vols, 1.15 eq.). The reaction slurry is heated to 70° C. for typically 3 hours when the reaction is complete (by HPLC analysis). The mixture is a thick green slurry at this point which is treated with THF (15 vols) to dissolve the product that has precipitated, leaving only the Pd/C catalyst out of solution. The mixture is then filtered hot through GFA filter to remove the catalyst. The vessel is rinsed with IMS (1 vol) and the wash used to rinse catalyst bed. A solution of p-toluenesulfonic acid monohydrate (1.50 wt, 4.0 eq.) in water (1.5 vols) is added to the filtered solution over 5 minutes at 65° C. The reaction solution is cooled to 60° C., with crystallization observed at 60-65° C. The resultant slurry is then stirred for at least 1 hour at 60° C. and then cooled to 20-25° C. and then held at this temperature for a further 1 hour. The product is isolated by filtration and the cake washed with IMS (3 vols). The product may be stored as a wet cake or dried.

Stage 3—5-{4-[(3-Chloro-4-{[(3-fluorophenyl)methyl]oxy}phenyl)amino]-6-quinazolinyl}-2-furancarbaldehyde 4-methylbenzenesulfonate (1 wt) and 2-(methylsulfonyl)ethylamine hydrochloride (0.4 wt, 1.60 equiv.) are suspended in THF (10 vols). Sequentially, acetic acid (0.354 vol., 4.00 equiv.) and di-isopropylethylamine (DIPEA, 1.08 vols, 4.00 equiv.) are added. The resulting solution is stirred at 30°-35° C. for ca. 1 hour then cooled to ca. 22° C. Sodium tri-acetoxyborohydride (0.66 wt, 2.00 equiv.) is then added. The resulting mixture is stirred at ca. 22° C. for 2-4 hours then sampled for HPLC analysis. The reaction is quenched by addition of aqueous sodium hydroxide (25% w/w, 3 vols.) followed by water (2 vols.). The aqueous phase is then separated, extracted with THF (2 vols) and the combined THF extracts are then washed twice with 25% w/v aqueous ammonium chloride solution (2×5 vols). A solution of p-toluenesulfonic acid monohydrate (p-TSA, 0.74 wt, 2.5 equiv.) in water (1 vol) is prepared, warmed to ca. 60° C., and N-(3-chloro-4-{[(3-fluorophenyl)methyl]oxy}phenyl)-6-[5-({[2-(methylsulfonyl)ethyl]amino}methyl)-2-furanyl]-4-quinazolinamine 4-methylbenzenesulfonate hydrate seeds are added. The THF solution of the free base is added to the p-TSA solution over at least 1 hr, maintaining the batch temperature at 60±3° C. The resulting suspension is stirred at ca. 60° C. for 1-2 hours, cooled to 20-25° C. over an hour and aged at this temperature for ca. 1 hr. The solid is collected by filtration, washed with 95:5 THF:Water (3×2 vols) and dried in vacuum at ca. 35° C. to give N-(3-chloro-4-{[(3-fluorophenyl)methyl]oxy}phenyl)-6-[5-({[2-(methylsulfonyl)ethyl]amino}methyl)-2-furanyl]-4-quinazolinamine 4-methylbenzenesulfonate hydrate as a bright yellow crystalline solid.

Stage 4—A slurry of N-(3-chloro-4-{[(3-fluorophenyl)methyl]oxy}phenyl)-6-[5-({[2-(methylsulfonyl)ethyl]amino}methyl)-2-furanyl]-4-quinazolinamine 4-methylbenzenesulfonate hydrate (1.00 rel. wt) in aqueous tetrahydrofuran (80:20 THF:Water, 17 vols.) is heated to 63-64° C. and held for at least 30 min until a solution forms. The solution is clarified while hot and a line wash applied (80:20 THF:Water, 0.5 vol.). THF (15.5 vols) is added over ca. 1 hour whilst maintaining the temperature at 60-63° C. and the solution seeded with GW572016F (0.002 rel. wt). The batch is maintained at 60-63° C. for at least 30 minutes to allow crystallization to become established. The batch is cooled to ca. 5° C. over ca. 2 hours and the product isolated by filtration. It is washed twice with aqueous THF (90:10 THF: Water, 2×2 vols.) followed once with aqueous THF (19:1 THF:Water, 1×2 vols.). The batch is dried under vacuum up to 45° C. to give N-(3-chloro-4-{[(3-fluorophenyl)methyl]oxy}phenyl)-6-[5-({[2-(methylsulfonyl)ethyl]amino}methyl)-2-furanyl]-4-quinazolinamine 4-methylbenzenesulfonate hydrate as a bright yellow crystalline solid.

### Example 3

### Preparation of (4-(3-Fluoro-benzyloxy)-3-bromophenyl)-(6-(5-((2-methanesulphonyl-ethylamino)-methyl)-furan-2-yl)quinazolin-4-yl)-amine Ditosylate (The Ditosylate Salt of the Compound of Formula (III″)

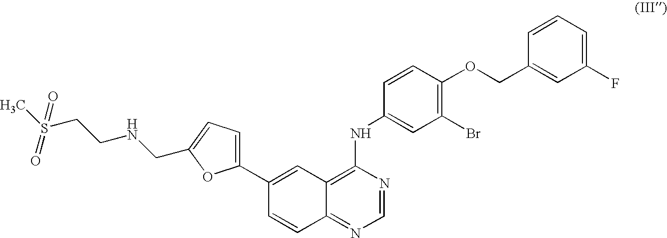

The H Cl salt of 5-(4-[3-bromo-4-(3-fluorobenzyloxy)-anilino]-6-quinazolinyl)-furan-2-carbaldehyde (prepared according to Procedure C, page 56 of WO 99/35146) was converted to the tosylate salt according to the procedure of Example 1, Stage 2. The resultant furan 2-carbaldehyde tosylate product was used to prepare the (4-(3-Fluoro-benzyloxy)-3-bromophenyl)-(6-(5-((2-methanesulphonyl-ethylamino)methyl)-furan-2-yl)quinazolin-4-yl)-amine ditosylate according to the procedure of Example 1, stage 3.

### Example 4

### Preparation of (4-(3-Fluoro-benzyloxy)-3-chlorophenyl)-(6-(2-((2-methanesulphonyl-ethylamino)-methyl)-thiazol-4-yl)quinazolin-4-yl)-amine Ditosylate (Ditosylate Salt of the Compound of Formula III′)

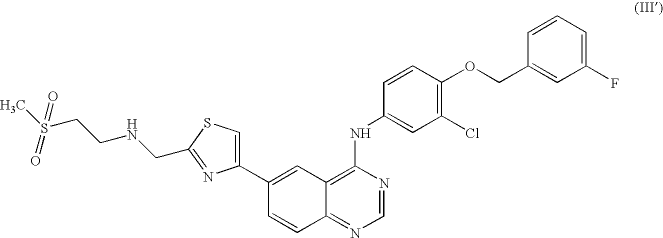

The HCL salt of (4-(3-Fluoro-benzyloxy)-3-chlorophenyl)-(6-(2-((2-methanesulphonyl-ethylamino)-methyl)-thiazol-4-yl)quinazolin-4-yl)-amine was prepared according to Procedure F, pages 57-59 of WO 99/35146 and then converted to the (4-(3-Fluoro-benzyloxy)-3-chlorophenyl)-(6-(2-((2-methanesulphonyl-ethylamino)-methyl)-thiazol-4-yl)quinazolin-4-yl)-amine ditosylate salt according to the procedures of Example 1.

### Example 5

### Preparation of Pyrollo-Pyrimidine IGF-1R Inhibitors

a) Compounds 5(a) and 5(b) were prepared according to methods similar to that described PCT Application PCT/EP02/05239 filed May 13, 2002 and published as International Application WO 02092599 on Nov. 21, 2002, and were characterized as the desired compounds 5(a) and 5(b).

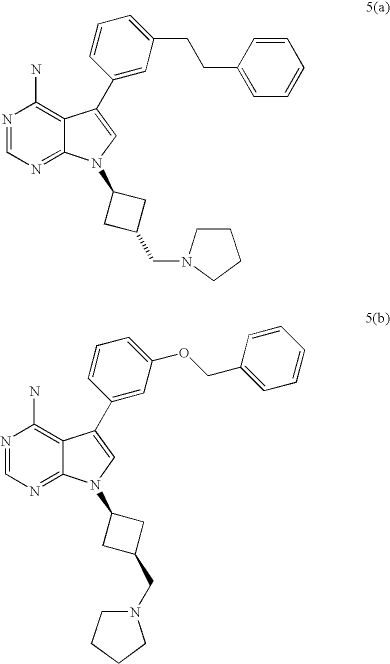

### Example 6

### Methods

GW572016 is N-{3-Chloro-4-[(3-fluorobenzyl)oxy]phenyl}-6-[5-({[2-(methane sulphonyl)ethyl]amino}methyl)-2-furyl]-4-quinazolinamine ditosylate monohydrate.

GW589522 is (4-(3-Fluoro-benzyloxy)-3-bromophenyl)-(6-(5-((2-methanesulphonyl-ethylamino)-methyl)-furan-2-yl)quinazolin-4-yl)-amine.

GW583340 is (4-(3-Fluoro-benzyloxy)-3-chlorophenyl)-(6-(2-((2-methanesulphonyl-ethylamino)-methyl)-thiazol-4-yl)quinazolin-4-yl)-amine.

GSK552602A is compound 5(a) described in Example 5.

GSK621659A is compound 5(b) described in Example 5.

A549 cells are non-small cell lung carcinoma cells obtainable from the American Type Culture Collection.

Colo205 cells are colon adenocarcinoma cells obtainable from the American Type Culture Collection.

MDA468 cells are MDA-MB-468 human breast adenocarcinoma cells obtainable from the American Type Culture Collection.

BT474 cells are human breast adenocarcinoma cells obtainable from the American Type Culture Collection.

T47D cells are human breast ductal carcinoma cells originally obtained from the American Type Culture Collection.

HN5 cells are LICRON-HN5 head and neck carcinoma cells, which were a gift from the Institute of Cancer Research, Surrey, U.K.

SCC15 cells are head and neck squamous cell carcinoma cells obtainable from the American Type Culture Collection.

H322 cells are NCI-H322 non-small cell lung carcinoma cells obtainable from the American Type Culture Collection.

H1299 cells are NCI-H1299 non-small cell lung carcinoma cells obtainable from the American Type Culture Collection.

SKOV3 cells are ovarian carcinoma cells obtainable from the American Type Culture Collection.

BxPC3 cells are pancreatic adenocarcinoma cells obtainable from the American Type Culture Collection.

For Examples 7-9 the following methods were utilized:

A549 cells were grown in RPMI-1640 supplemented with 25 mM HEPES, 10 mM glutamine and 10% fetal bovine serum and maintained at 37° C. and 5% CO₂ in a humid incubator. Colo205 and MDA-MB-468 cells were grown in DMEM supplemented with 25 mM HEPES, 10 mM glutamine, 4.5 g/L d-glucose, and 10% fetal bovine serum. Assays were performed in 96 well microtiter plates with optimum seeding densities for each cell line.

Apoptosis was measured using the Roche Cell Death ELISA^{Plus} kit (catalog 1 774 425) which detects fragmented nucleosomal DNA that is generated during apoptosis. A second assay was used to demonstrate caspase activation (Promega Apo-ONE™ Homogeneous Caspase-3/7 Assay, catalog G7791) which is an early event in the apoptotic cascade.

Cell proliferation was measured using the Celltiter Glo® Luminescent Cell Viability Assay available from Promega Corporation and the Roche BrdU Cell Proliferation ELISA assay.

### Example 7

### Dosing A549 Cells with GW572016 and the IGF-1R Inhibitor GSK621659A

GW572016 (GW2016) and GSK621659A (GSK1659) alone and in a 1:1 molar ratio were coincubated with A549 cells for 24 h. Cell death was measured using the Roche Cell Death ELISA^{Plus} kit and Promega Apo-ONE™ Homogeneous Caspase-3/7 Assay. Total cell number was measured using the Celltiter Glo® Luminescent Cell Viability Assay and cell proliferation was measured with the Roche BrdU Cell Proliferation ELISA assay.

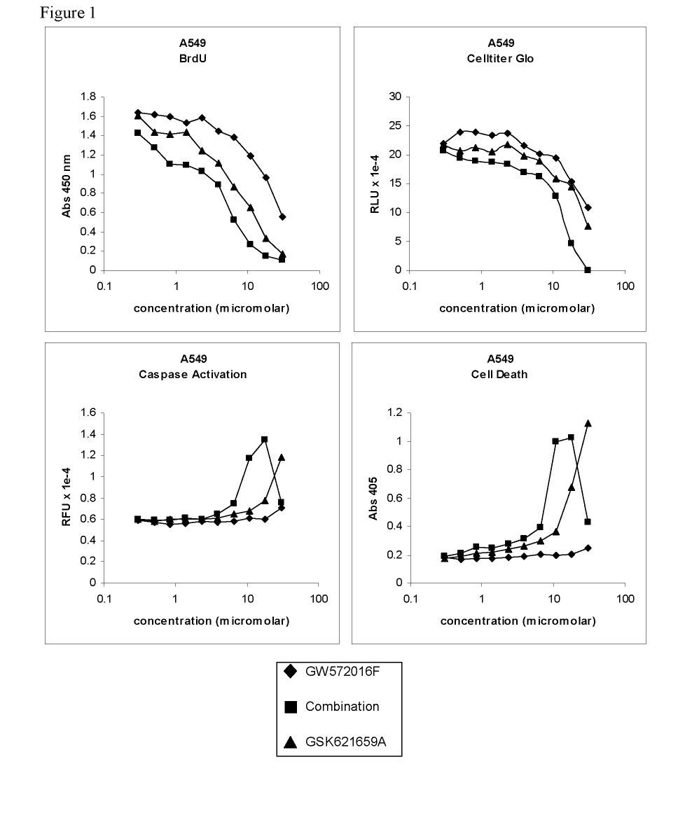

The results for each assay are shown in the 4 plots of FIG. 1. Treatment of the A549 tumor cells with GW572016 and GSK621659A resulted in decreased cell proliferation and increased apoptosis. The combination of both agents was more effective than either agent alone with regards to both proliferation and apoptosis.

### Example 8

### Dosing Colo205 Cells with GW572016 and the IGF-1R Inhibitor GSK621659A

GW572016 (GW2016) and GSK621659A (GSK1659) alone and in a 1:1 molar ratio were coincubated with Colo205 cells for 24 h. Cell death was measured using the Roche Cell Death ELISA^{Plus} kit and Promega Apo-ONE™ Homogeneous Caspase-3/7 Assay. Total cell number was measured using the Celltiter Glo® Luminescent Cell Viability Assay and cell proliferation was measured with the Roche BrdU Cell Proliferation ELISA assay.

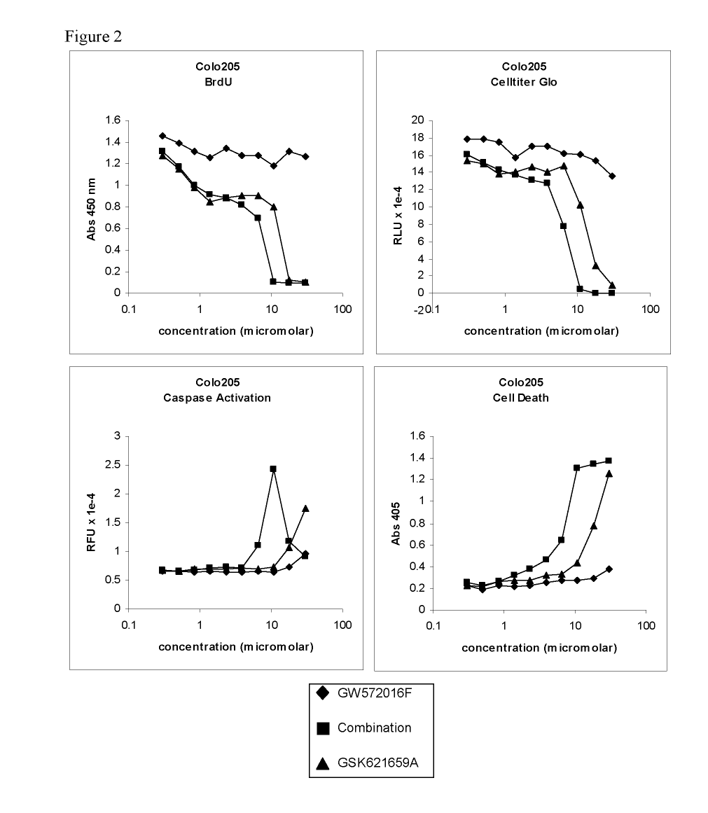

The results for each assay are shown in the 4 plots of FIG. 2. Treatment of the Colo205 tumor cells with GW572016 and GSK621659A resulted in decreased cell proliferation and increased apoptosis. The combination of both agents was more effective than either agent alone with regards to both proliferation and apoptosis.

### Example 9

### Dosing MDA-MB-468 Cells with GW572016 and the IGF-1R Inhibitor GSK621659A

GW572016 (GW2016) and GSK621659A (GSK1659) alone and in a 1:1 molar ratio were coincubated with MDA-MB-468 cells for 24 h. Cell death was measured using the Roche Cell Death ELISA^{Plus} kit and Promega Apo-ONE™ Homogeneous Caspase-3/7 Assay. Total cell number was measured using the Celltiter Glo® Luminescent Cell Viability Assay and cell proliferation was measured with the Roche BrdU Cell Proliferation ELISA assay.

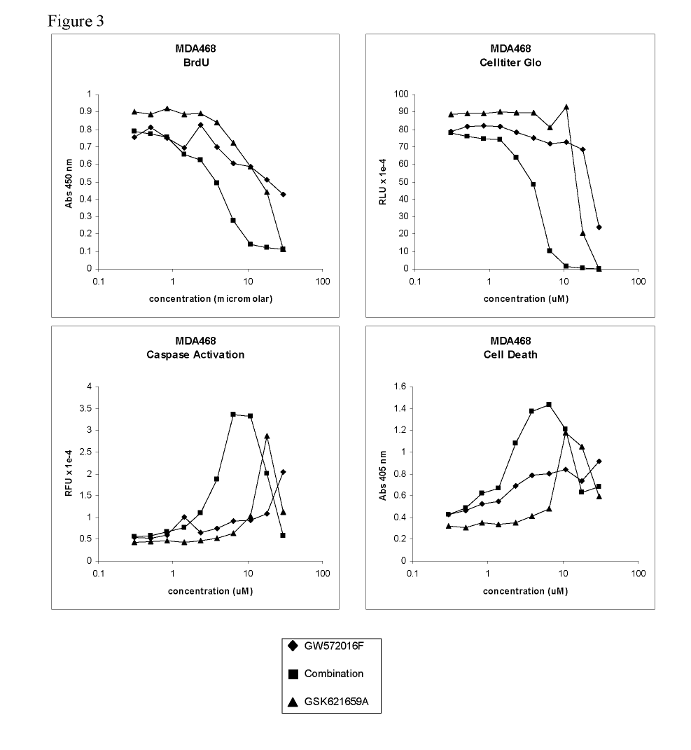

The results for each assay are shown in the 4 plots of FIG. 3. Treatment of the MDA-MB-468 tumor cells with GW572016 and GSK621659A resulted in decreased cell proliferation and increased apoptosis. The combination of both agents was more effective than either agent alone with regards to both proliferation and apoptosis.

### Example 10

### Dosing of Various Cancer Cell Lines with GW572016 and the IGF-1R Inhibitors GSK621659A, GSK552602, and Alpha-IR₃ Antibody

Human tumor cell lines from breast: MDA468, BT474 and T47D; head/neck: HN5 and SCC15; lung: A549, H322 and H1299, colon: Colo205; ovary: SKOV3; and pancreas: BxPC3 were cultured in a humidified incubator at 37° C. in 95% air, 5% CO₂ in the following media: MDA468, HN5 and Colo205, Dulbecco's modified Eagle medium (DMEM) containing 10% fetal bovine serum (FBS); A549, H322, H1299, BxPC3, BT474, T47D, SKOV3 and SCC15, RPMI 1640 containing 10% FBS. Cells were assayed in a 96-well tissue culture plate (Falcon 3075) with the following plating densities: HN5 3,000 cells/well; A549, 4,000 cells/well; H1299, SKOV3 and T47D, 5,000 cells/well; BT474, MDA468, Colo205, SCC15, H322 and BxPC3, 10,000 cells/well. Approximately 24 hours after plating, cells were exposed to compounds, cells were treated with ten, two-fold serial dilutions of GSK552602A or GSK621659A, GW572016 or the combination of the two agents at concentrations resulting in complete dose response curves of each compound. For the antibody, alpha-IR3, cells were treated with ten, two-fold serial dilutions with a concentration range from 10 to 0.02 micrograms/ml. Cells were incubated in the presence of compound and/or antibody for 3 days. Media were then removed by aspiration. Cell biomass was estimated by staining cells at room temperature for at least 30 minutes with 90 microliters per well methylene blue (Sigma M9140, 0.5% in 1:1 ethanol:water). Stain was removed, and the plates rinsed by immersion in deionized water and air-dried. To release stain from the cells, 100 microliters of solubilization solution was added (1% N-lauroyl sarcosine, Sodium salt, Sigma L5125, in PBS), and plates were shaken gently for about 30 minutes. Optical density at 620 nM was measured on a microplate reader. Cell growth was calculated relative to vehicle treated control wells. Concentration of compound that inhibits 50% of control cell growth (IC₅₀) was interpolated using nonlinear regression and the equation, y=Vmax*(1−(x/(K+x)))+Y2. Combination Index values were generated by inserting the interpolated IC₅₀ values in to the mutually non-exclusive equation derived by Chou and Talalay: CI=Dₐ/IC₅₀₍ₐ₎+D_{b}/IC_{50(b)}+(Dₐ*D_{b})/(IC₅₀₍ₐ₎*IC_{50(b)}), where IC₅₀₍ₐ₎ was the IC₅₀ of lapatinib, IC_{50(b)} was the IC₅₀ for the IGF-1R inhibitor, Dₐ was the concentration of lapatinib in combination with the IGF-1R inhibitor that inhibited 50% of cell growth and D_{b} was the concentration of the IGF-1R inhibitor in combination with lapatinib that inhibited 50% of cell growth. When the CI value was between 0.9 and 1.10, the combination of the two agents was deemed additive. Combination Index values less than 0.9 were considered indicative of synergy. Combination Index values>1.10 indicated antagonism.

For investigation of the induction of apoptosis, MDA468, Colo205, A549 and H322 cells were plated at 5,000 cells per well in a 96-well tissue culture plate and allowed to attach for approximately 24 hours. Cells were then treated with compounds as described above. After 24 hours of compound treatment, levels of apoptosis were estimated using the Roche Cell Death ELISA (Cat. No. 11 774 425 001) following the instructions provided by the manufacturer.

### Summary and Results:

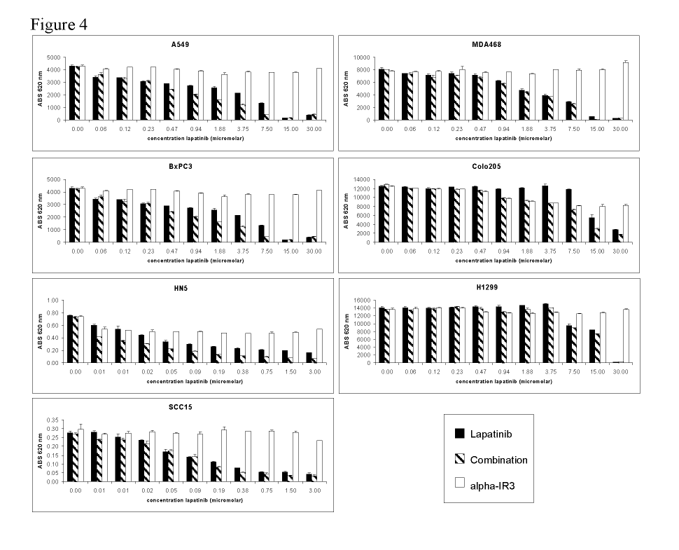

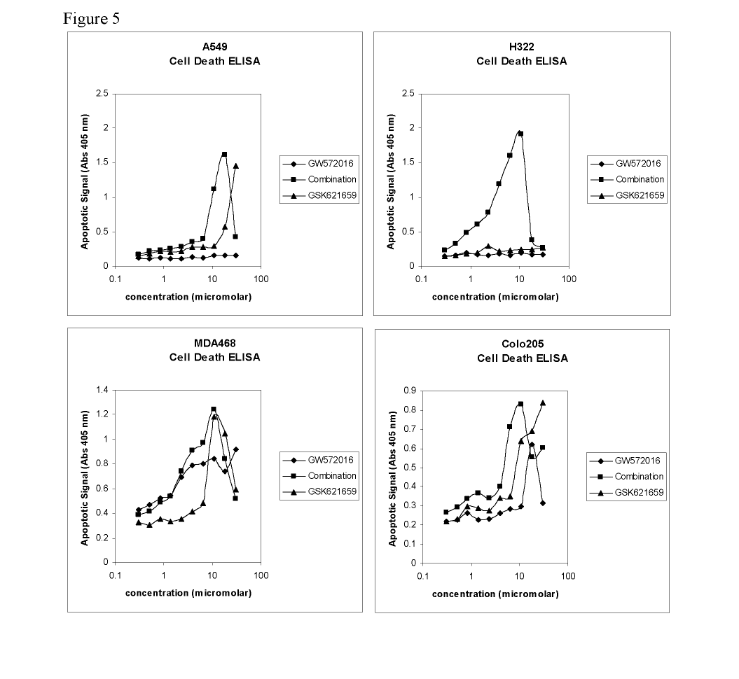

The combination of GW572016 and inhibitors of insulin-like growth factor one receptor (IGF-1R) was investigated in cell-based assays. The agents used for targeting IGF-1R included the small molecule inhibitors GSK552602, GSK621659 (Table 1) or the antibody, alpha-IR3. GSK552606 and GSK621659 also inhibit the insulin receptor. Alpha-IR3 is selective for IGF-1R and does not cross-react with the insulin receptor. Eleven cell lines from lung, colon, breast, head/neck, ovary and pancreas tumor tissue were tested for their response to the combination of lapatinib and at least one of the small molecule inhibitors. Seven of these lines were tested for their response to the combination of lapatinib and alpha-IR3. Inhibition of cell growth was estimated by staining cells with methylene blue after three days of treatment with compounds or vehicle (DMSO). As described above, Combination index (CI) values were generated using a modification of the method of Chou and Talalay. In addition, four of the tumor cell lines were investigated for induction of apoptosis in response to the combination of GSK621659 and lapatinib. The data indicated a synergistic response to the combination of the IGF-1R small-molecule inhibitors and lapatinib in the seven of the eleven cell lines tested: A549, H322, BXPC3, HN5, SCC15, SKOV3 and T47D cell lines. A response that was approximately additive was observed in the MDA468, Colo205 and H1299 cell lines. The results for the BT474 cell line varied, with one test indicating antagonism and one test indicating slight synergy (Table 2). Due to the weak response of the cell lines to alpha-IR3 as a monotherapy, Cl values could not be determined for the combination of lapatinib and alpha-IR3. Based on the graphs in FIG. 4, the results for the combination of lapatinib and alpha-IR3 were consistent with the combination of lapatinib and the small-molecule inhibitors, with A549, H322, BxPC3 and HN5 showing a better response to the combination of lapatinib and alpha-IR3 than to either agent as a monotherapy. Experiments investigating induction of apoptosis (FIG. 5) indicate a strong increase in apoptotic signal in cells treated with the combination of lapatinib and GSK621659 relative to either monotherapy in the two lung cell lines, A549 and H322. The MDA468 breast cell line and the Colo205 colon cell line responded to the combination of lapatinib and GSK621659 with a slight increase in apoptosis relative to the monotherapies, consistent to the additive response observed in these two lines in the cell growth assay.

TABLE 1
Compound Structures and activities

| Compound | Enzyme activities |
|---|---|
| 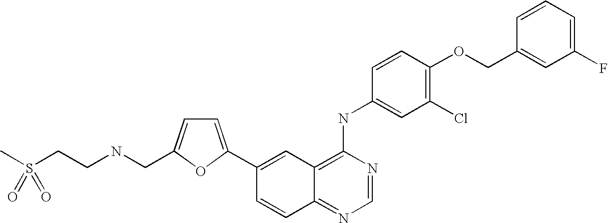 | EGFR KI = 3 nM ErbB2 Ki = 13 nM |
| GW572016 | |
| 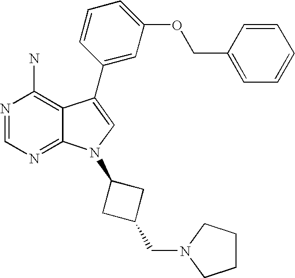 | IGF-1R IC₅₀ = 20 nM Insulin Receptor IC₅₀ = 50 nM |
| GSK552602A | |
| 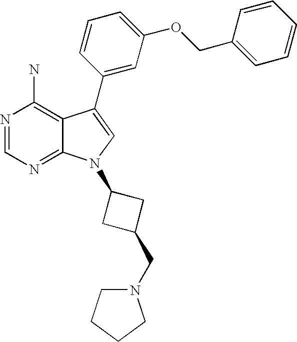 | IGF-1R IC₅₀ = 25 nM Insulin Receptor IC₅₀ = 63 nM |
| GSK621659A | |

TABLE 2
CI values from multiple experiments combining lapatinib and the
small molecule IGF-1R inhibitors, GSK552602 and GSK621659.

| Cell | Tissue of | CI | CI |
|---|---|---|---|
| Line | origin | GSK552602A | GSK621659A |
| A549 | Lung | 0.19 +/− 0.00 (n = 3) | 0.23 +/− 0.03 (n = 6) |
| H322 | Lung | NT | 0.28 +/− 0.04 (n = 7) |
| H1299 | Lung | 1.10 +/− 0.16 (n = 3) | NT |
| BxPC3 | Pancreas | 0.24, 0.20 | NT |
| Colo205 | Colon | 1.05, 1.03 | NT |
| MDA468 | Breast | 1.10 +/− 0.07 (n = 3) | NT |
| T47D | Breast | NT | 0.36, 0.21 |
| BT474 | Breast | NT | 3.4, 0.88 |
| SKOV3 | Ovary | NT | 0.39 |
| SCC15 | Head/neck | NT | 0.45 |
| HN5 | Head/neck | 0.23 | 0.36 +/− 0.04 (n = 4) |

NT = not tested
Averages +/−95% confidence limit where n ≧ 3

What is claimed is:

1: A method of treating a susceptible cancer in a mammal, comprising: administering to said mammal therapeutically effective amounts of (i) a compound of formula (I) 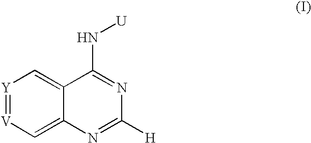
or a salt or solvate thereof;
wherein
Y is CR¹ and V is N;
or Y is CR¹ and V is CR²;
R¹ represents a group CH₃SO₂CH₂CH₂NHCH₂—Ar—, wherein Ar is selected from phenyl, furan, thiophene, pyrrole and thiazole, each of which may optionally be substituted by one or two halo, C₁₋₄ alkyl or C₁₋₄ alkoxy groups;
R² is selected from the group comprising hydrogen, halo, hydroxy, C₁₋₄ alkyl, C₁₋₄ alkoxy, C₁₋₄ alkylamino and di[C₁₋₄ alkyl]amino;
U represents a phenyl, pyridyl, 3H-imidazolyl, indolyl, isoindolyl, indolinyl, isoindolinyl, 1H-indazolyl, 2,3-dihydro-1H-indazolyl, 1H-benzimidazolyl, 2,3-dihydro-1H-benzimidazolyl or 1H-benzotriazolyl group, substituted by an R³ group and optionally substituted by at least one independently selected R⁴ group;
R³ is selected from a group comprising benzyl, halo-, dihalo- and trihalobenzyl, benzoyl, pyridylmethyl, pyridylmethoxy, phenoxy, benzyloxy, halo-, dihalo- and trihalobenzyloxy and benzenesulphonyl;
or R³ represents trihalomethylbenzyl or trihalomethylbenzyloxy;
or R³ represents a group of formula 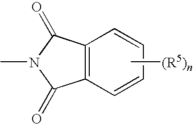
wherein each R⁵ is independently selected from halogen, C₁₋₄ alkyl and C₁₋₄ alkoxy; and n is 0 to 3;
each R⁴ is independently hydroxy, halogen, C₁₋₄ alkyl, C₂₋₄ alkenyl, C₂₋₄ alkynyl, C₁₋₄ alkoxy, amino, C₁₋₄ alkylamino, di[C₁₋₄ alkyl]amino, C₁₋₄ alkylthio, C₁₋₄ alkylsulphinyl, C₁₋₄ alkylsulphonyl, C₁₋₄ alkylcarbonyl, carboxy, carbamoyl, C₁₋₄ alkoxycarbonyl, C₁₋₄ alkanoylamino, N—(C₁₋₄ alkyl)carbamoyl, N,N-di(C₁₋₄ alkyl)carbamoyl, cyano, nitro and trifluoromethyl; and
(ii) at least one IGF-1R inhibitor.

2: A method of treating a susceptible cancer in a mammal, comprising: administering to said mammal therapeutically effective amounts of (i) a compound of formula (II): 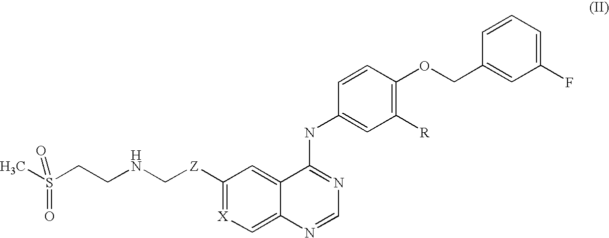
or a salt or solvate thereof, wherein R is —Cl or —Br, X is CH, N, or CF, and Z is thiazole or furan; and
(ii) at least one IGF-1R inhibitor.

3: A method of treating a susceptible cancer in a mammal, comprising: administering to said mammal therapeutically effective amounts of (i) a compound of formula (III): 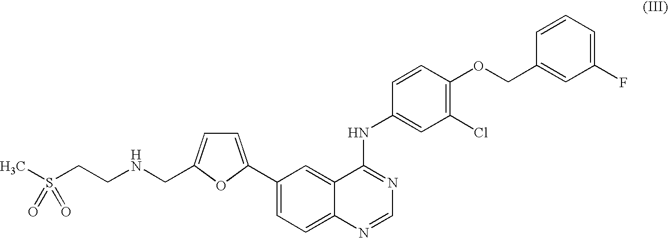
or a salt or solvate thereof; and
(ii) at least one IGF-1R inhibitor.

4: A cancer treatment combination, comprising: therapeutically effective amounts of (i) a compound of formula (I) 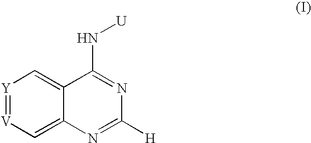
or a salt or solvate thereof;
wherein
Y is CR¹ and V is N;
or Y is CR¹ and V is CR²;
R¹ represents a group CH₃SO₂CH₂CH₂NHCH₂—Ar—, wherein Ar is selected from phenyl, furan, thiophene, pyrrole and thiazole, each of which may optionally be substituted by one or two halo, C₁₋₄ alkyl or C₁₋₄ alkoxy groups;
R² is selected from the group comprising hydrogen, halo, hydroxy, C₁₋₄ alkyl, C₁₋₄ alkoxy, C₁₋₄ alkylamino and di[C₁₋₄ alkyl]amino;
U represents a phenyl, pyridyl, 3H-imidazolyl, indolyl, isoindolyl, indolinyl, isoindolinyl, 1H-indazolyl, 2,3-dihydro-1H-indazolyl, 1H-benzimidazolyl, 2,3-dihydro-1H-benzimidazolyl or 1H-benzotriazolyl group, substituted by an R³ group and optionally substituted by at least one independently selected R⁴ group;
R³ is selected from a group comprising benzyl, halo-, dihalo- and trihalobenzyl, benzoyl, pyridylmethyl, pyridylmethoxy, phenoxy, benzyloxy, halo-, dihalo- and trihalobenzyloxy and benzenesulphonyl;
or R³ represents trihalomethylbenzyl or trihalomethylbenzyloxy;
or R³ represents a group of formula 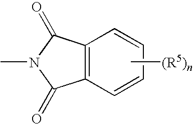
wherein each R⁵ is independently selected from halogen, C₁₋₄ alkyl and C₁₋₄ alkoxy; and n is 0 to 3;
each R⁴ is independently hydroxy, halogen, C₁₋₄ alkyl, C₂₋₄ alkenyl, C₂₋₄ alkynyl, C₁₋₄ alkoxy, amino, C₁₋₄ alkylamino, di[C₁₋₄ alkyl]amino, C₁₋₄ alkylthio, C₁₋₄ alkylsulphinyl, C₁₋₄ alkylsulphonyl, C₁₋₄ alkylcarbonyl, carboxy, carbamoyl, C₁₋₄ alkoxycarbonyl, C₁₋₄ alkanoylamino, N—(C₁₋₄ alkyl)carbamoyl, N,N-di(C₁₋₄ alkyl)carbamoyl, cyano, nitro and trifluoromethyl; and
(ii) at least one IGF-1R inhibitor.

5: A cancer treatment combination, comprising: therapeutically effective amounts of (i) a compound of formula (II): 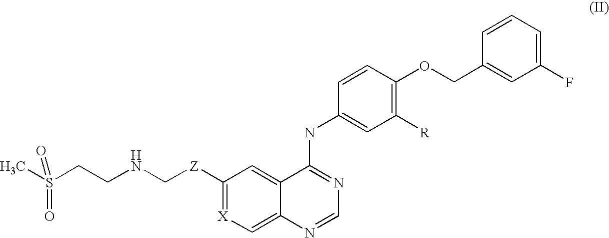
or a salt or solvate thereof, wherein R is —Cl or —Br, X is CH, N, or CF, and Z is thiazole or furan; and
(ii) at least one IGF-1R inhibitor.

6: A cancer treatment combination, comprising: therapeutically effective amounts of (i) a compound of formula (III): 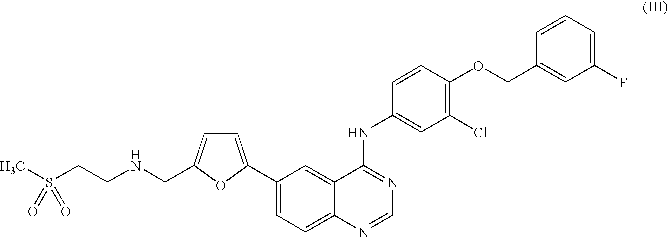
or a salt or solvate thereof; and
(ii) at least one IGF-1R inhibitor.
